(19) United States
(12) Patent Application Publication
(10) Pub. No.: US 2025/0177033 A1
(43) Pub. Date: Jun. 5, 2025

(54) BLADE RETENTION SYSTEMS FOR SURGICAL TOOL END EFFECTORS

(71) Applicant: Cilag GmbH International, Zug (CH)

(72) Inventors: John A. HIBNER, Mason, OH (US); Shinnosuke INOUE, Daly City, CA (US); Samuel Gerard SELLE, Milpitas, CA (US); Thomas B. REMM, Cincinnati, OH (US)

(73) Assignee: Cilag GmbH International, Zug (CH)

(21) Appl. No.: 18/525,294

(22) Filed: Nov. 30, 2023

(51) Int. Cl. A61B 18/14; A61B 17/00; A61B 17/3201; A61B 18/12; A61B 34/00; A61B 34/30

(52) U.S. Cl. CPC A61B 18/1445; A61B 17/3201; A61B 34/30; A61B 34/71; A61B 2017/00469; A61B 2017/00526; A61B 2018/1253; A61B 2034/305

(57) ABSTRACT

A surgical tool includes a drive housing, an elongate shaft extending distally from the drive housing, a wrist arranged at a distal end of the shaft, and an end effector operatively coupled to the wrist and including first and second blade pulleys rotatably mounted to the wrist, first and second blade holders mounted to the first and second blade pulleys, respectively, and cooperatively defining an interior, first and second blades mountable to the first and second blade holders, respectively, such that rotational movement of the first and second blade pulleys correspondingly moves the first and second blades between closed and open positions, and a blade retention system that releasably secures the first and second blades to the first and second blade holders, respectively, and allows a user to manually remove and replace the first and second blades.

### BACKGROUND

Minimally invasive surgical (MIS) instruments are often preferred over traditional open surgical devices due to reduced post-operative recovery time and minimal scarring. Laparoscopic surgery is one type of MIS procedure in which one or more small incisions are formed in the abdomen of a patient and a trocar is inserted through the incision to form a pathway that provides access to the abdominal cavity. Through the trocar, a variety of instruments and surgical tools can be introduced into the abdominal cavity. The instruments and tools introduced into the abdominal cavity via the trocar can be used to engage and/or treat tissue in a number of ways to achieve a diagnostic or therapeutic effect.

Various robotic systems have been developed to assist in MIS procedures. Robotic systems can allow for more instinctive hand movements by maintaining natural eye-hand axis. Robotic systems can also allow for more degrees of freedom in movement by including an articulable “wrist” joint that creates a more natural hand-like articulation. In such systems, an end effector positioned at the distal end of the instrument can be articulated (moved) using a cable driven motion system having one or more drive cables that extend through the wrist joint. A user (e.g., a surgeon) is able to remotely operate the end effector by grasping and manipulating in space one or more controllers that communicate with a tool driver coupled to the surgical instrument. User inputs are processed by a computer system incorporated into the robotic surgical system, and the tool driver responds by actuating the cable driven motion system. Moving the drive cables articulates the end effector to desired angular positions and configurations.

Some end effectors include high-wear components that can mechanically or physically degrade over time and thereby limit the useful life of the end effector. One example of a high-wear component is the blades of surgical scissors, which can dull over time, and thereby adversely affect the efficiency of the end effector. Moreover, when surgical scissors are energized, such as in monopolar scissor end effectors, the useful life of the blades can be decreased by the degradation of the cutting edge of the blades due to the monopolar energy used during procedures.

What is needed is a method and system of more easily replacing the blades of surgical scissor end effectors.

### BRIEF DESCRIPTION OF THE DRAWINGS

The following figures are included to illustrate certain aspects of the present disclosure, and should not be viewed as exclusive embodiments. The subject matter disclosed is capable of considerable modifications, alterations, combinations, and equivalents in form and function, without departing from the scope of this disclosure.

FIG. 1 is a block diagram of an example robotic surgical system that may incorporate some or all of the principles of the present disclosure.

FIG. 2 is an isometric side view of an example surgical tool that may incorporate some or all of the principles of the present disclosure.

FIG. 3 illustrates potential degrees of freedom in which the wrist of the surgical tool of FIG. 2 may be able to articulate (pivot) and translate.

FIGS. 4A-4C are enlarged isometric views of the distal end of the surgical tool of FIG. 2, according to one or more embodiments.

FIGS. 5A-5C are enlarged isometric views of the distal end of the surgical tool of FIG. 2, according to one or more additional embodiments.

FIGS. 6A-6C are enlarged isometric views of the distal end of the surgical tool of FIG. 2, according to one or more additional embodiments.

FIGS. 7A-7D are enlarged isometric views of the distal end of the surgical tool of FIG. 2, according to one or more additional embodiments.

FIGS. 8A and 8B are enlarged isometric views of the distal end of the surgical tool of FIG. 2, according to one or more additional embodiments.

FIG. 9 is an isometric, exploded view of an example the blades of FIGS. 8A-8B, according to one or more embodiments.

FIGS. 10A and 10B are isometric and end views of the surgical tool showing before and after installation of the blades, respectively, according to one or more embodiments.

FIGS. 11A and 11B are left and right isometric views, respectively, of an example blade installation and removal tool, according to one or more embodiments of the present disclosure.

FIGS. 12A-12D depict progressive steps in installing the blades using the blade installation and removal tool of FIGS. 11A-11B, according to one or more embodiments of the present disclosure.

FIGS. 13A-13C depict progressive steps in removing the blades using the blade installation and removal tool of FIGS. 11A-11B, according to one or more embodiments of the present disclosure.

### DETAILED DESCRIPTION

The present disclosure is related to robotic surgical systems and, more particularly, to blade retention systems for end effectors that releasably secure blade to corresponding blade holders, while allowing a user to manually remove and replace the blades as needed.

The embodiments disclosed herein describe a surgical tool that includes a drive housing, an elongate shaft extending distally from the drive housing, a wrist arranged at a distal end of the shaft, and an end effector operatively coupled to the wrist. The end effector may include first and second blade pulleys rotatably mounted to the wrist, first and second blade holders mounted to the first and second blade pulleys, respectively, and cooperatively defining an interior, and first and second blades mountable to the first and second blade holders, respectively, such that rotational movement of the first and second blade pulleys correspondingly moves the first and second blades between closed and open positions. A blade retention system releasably secures the first and second blades to the first and second blade holders, respectively, and allows a user to manually remove and replace the first and second blades.

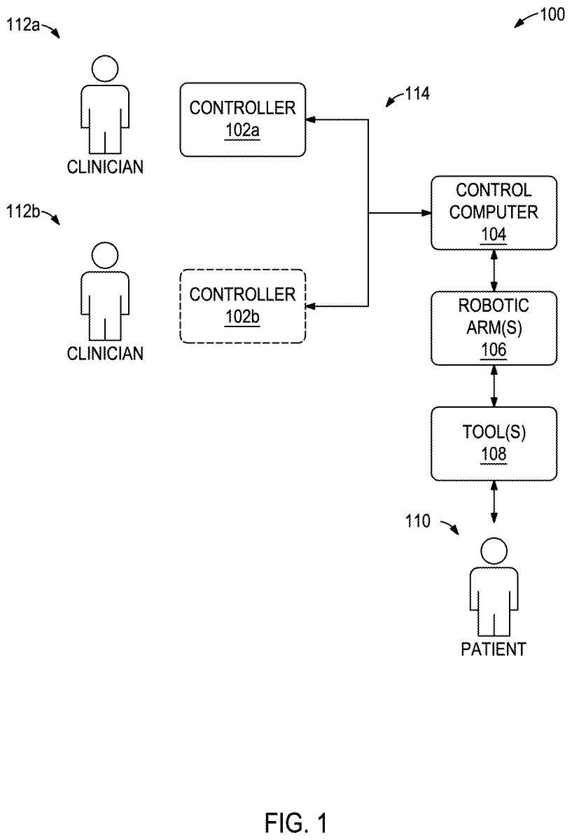

FIG. 1 is a block diagram of an example robotic surgical system 100 that may incorporate some or all of the principles of the present disclosure. As illustrated, the system 100 can include at least one set of user input controllers 102a and at least one control computer 104. The control computer 104 may be mechanically and/or electrically coupled to a robotic manipulator and, more particularly, to one or more robotic arms 106 (alternately referred to as “tool drivers”). In some embodiments, the robotic manipulator may be included in or otherwise mounted to an arm cart capable of making the system portable. Each robotic arm 106 may include and otherwise provide a location for mounting one or more surgical instruments or tools 108 for performing various surgical tasks on a patient 110. Operation of the robotic arms 106 and associated tools 108 may be directed by a clinician 112a (e.g., a surgeon) from the user input controller 102a.

In some embodiments, a second set of user input controllers 102b (shown in dashed line) may be operated by a second clinician 112b to direct operation of the robotic arms 106 and tools 108 via the control computer 104 and in conjunction with the first clinician 112a. In such embodiments, for example, each clinician 112a,b may control different robotic arms 106 or, in some cases, complete control of the robotic arms 106 may be passed between the clinicians 112a,b as needed. In some embodiments, additional robotic manipulators having additional robotic arms may be utilized during surgery on the patient 110, and these additional robotic arms may be controlled by one or more of the user input controllers 102a,b.

The control computer 104 and the user input controllers 102a,b may be in communication with one another via a communications link 114, which may be any type of wired or wireless telecommunications means configured to carry a variety of communication signals (e.g., electrical, optical, infrared, etc.) according to any communications protocol. In some applications, for example, there is a tower with ancillary equipment and processing cores designed to drive the robotic arms 106.

The user input controllers 102a,b generally include one or more physical controllers that can be grasped by the clinicians 112a,b and manipulated in space while the surgeon views the procedure via a stereo display. The physical controllers generally comprise manual input devices movable in multiple degrees of freedom, and which often include an actuatable handle for actuating the surgical tool(s) 108, for example, for opening and closing opposing jaws, applying an electrical potential (current) to an electrode, or the like. The control computer 104 can also include an optional feedback meter viewable by the clinicians 112a,b via a display to provide a visual indication of various surgical instrument metrics, such as the amount of force being applied to the surgical instrument (i.e., a cutting instrument or dynamic clamping member).

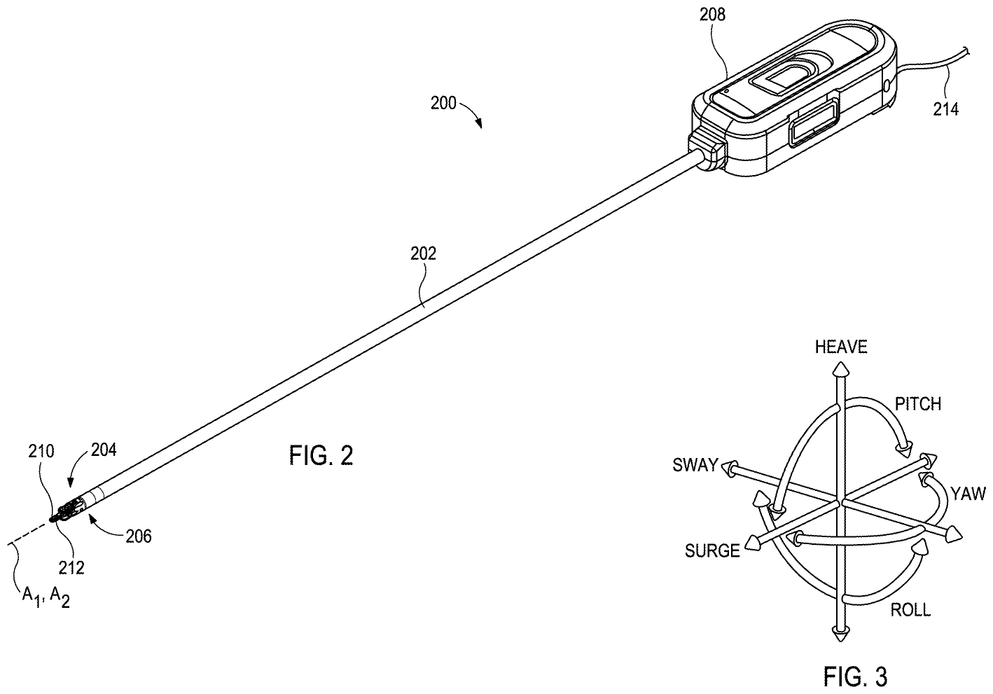

FIG. 2 is an isometric side view of an example surgical tool 200 that may incorporate some or all of the principles of the present disclosure. The surgical tool 200 may be the same as or similar to the surgical tool(s) 108 of FIG. 1 and, therefore, may be used in conjunction with a robotic surgical system, such as the robotic surgical system 100 of FIG. 1. Accordingly, the surgical tool 200 may be designed to be releasably coupled to a tool driver included in the robotic surgical system 100. In other embodiments, however, aspects of the surgical tool 200 may be adapted for use in a manual or hand-operated manner, without departing from the scope of the disclosure.

As illustrated, the surgical tool 200 includes an elongated shaft 202, an end effector 204, a wrist 206 (alternately referred to as a “wrist joint” or an “articulable wrist joint”) that couples the end effector 204 to the distal end of the shaft 202, and a drive housing 208 coupled to the proximal end of the shaft 202. In applications where the surgical tool is used in conjunction with a robotic surgical system (e.g., the robotic surgical system 100 of FIG. 1), the drive housing 208 can include coupling features that releasably couple the surgical tool 200 to the robotic surgical system.

The terms “proximal” and “distal” are defined herein relative to a robotic surgical system having an interface configured to mechanically and electrically couple the surgical tool 200 (e.g., the housing 208) to a robotic manipulator. The term “proximal” refers to the position of an element closer to the robotic manipulator and the term “distal” refers to the position of an element closer to the end effector 204 and thus further away from the robotic manipulator. Alternatively, in manual or hand-operated applications, the terms “proximal” and “distal” are defined herein relative to a user, such as a surgeon or clinician. The term “proximal” refers to the position of an element closer to the user and the term “distal” refers to the position of an element closer to the end effector 204 and thus further away from the user. Moreover, the use of directional terms such as above, below, upper, lower, upward, downward, left, right, and the like are used in relation to the illustrative embodiments as they are depicted in the figures, the upward or upper direction being toward the top of the corresponding figure and the downward or lower direction being toward the bottom of the corresponding figure.

During use of the surgical tool 200, the end effector 204 is configured to move (pivot) relative to the shaft 202 at the wrist 206 to position the end effector 204 at desired orientations and locations relative to a surgical site. To accomplish this, the housing 208 includes (contains) various drive inputs and mechanisms (e.g., gears, actuators, etc.) designed to control operation of various features associated with the end effector 204 (e.g., clamping, firing, cutting, rotation, articulation, etc.). In at least some embodiments, the shaft 202, and hence the end effector 204 coupled thereto, is configured to rotate about a longitudinal axis A₁ of the shaft 202. In such embodiments, at least one of the drive inputs included in the housing 208 is configured to control rotational movement of the shaft 202 about the longitudinal axis A₁.

The shaft 202 is an elongate member extending distally from the housing 208 and has at least one lumen extending therethrough along its axial length. In some embodiments, the shaft 202 may be fixed to the housing 208, but could alternatively be rotatably mounted to the housing 208 to allow the shaft 202 to rotate about the longitudinal axis A₁. In yet other embodiments, the shaft 202 may be releasably coupled to the housing 208, which may allow a single housing 208 to be adaptable to various shafts having different end effectors.

The end effector 204 can exhibit a variety of sizes, shapes, and configurations. In the illustrated embodiment, the end effector 204 comprises surgical scissors that includes opposing first (upper) and second (lower) blades 210, 212 configured to move (articulate) between open and closed positions. As will be appreciated, however, the blades 210, 212 may alternatively comprise opposing jaws that form part of other types of end effectors such as, but not limited to, a needle driver, a clip applier, a tissue grasper, a vessel sealer, a combination tissue grasper and vessel sealer, a babcock including a pair of opposed grasping jaws, bipolar jaws (e.g., bipolar Maryland grasper, forceps, a fenestrated grasper, etc.), etc. One or both of the blades 210, 212 may be configured to pivot to articulate the end effector 204 between the open and closed positions.

FIG. 3 illustrates the potential degrees of freedom in which the wrist 206 may be able to articulate (pivot) and thereby move the end effector 204. The wrist 206 can have any of a variety of configurations. In general, the wrist 206 comprises a joint configured to allow pivoting movement of the end effector 204 relative to the shaft 202. The degrees of freedom of the wrist 206 are represented by three translational variables (i.e., surge, heave, and sway), and by three rotational variables (i.e., Euler angles or roll, pitch, and yaw). The translational and rotational variables describe the position and orientation of the end effector 204 with respect to a given reference Cartesian frame. As depicted in FIG. 3, “surge” refers to forward and backward translational movement, “heave” refers to translational movement up and down, and “sway” refers to translational movement left and right. With regard to the rotational terms, “roll” refers to tilting side to side, “pitch” refers to tilting forward and backward, and “yaw” refers to turning left and right.

The pivoting motion can include pitch movement about a first axis of the wrist 206 (e.g., X-axis), yaw movement about a second axis of the wrist 206 (e.g., Y-axis), and combinations thereof to allow for 360° rotational movement of the end effector 204 about the wrist 206. In other applications, the pivoting motion can be limited to movement in a single plane, e.g., only pitch movement about the first axis of the wrist 206 or only yaw movement about the second axis of the wrist 206, such that the end effector 204 moves only in a single plane.

Referring again to FIG. 2, the surgical tool 200 may also include a plurality of drive cables (obscured in FIG. 2) that form part of a cable driven motion system configured to facilitate actuation and articulation of the end effector 204 relative to the shaft 202. Moving (actuating) one or more of the drive cables moves the end effector 204 between an unarticulated position and an articulated position. The end effector 204 is depicted in FIG. 2 in the unarticulated position where a longitudinal axis A₂ of the end effector 204 is substantially aligned with the longitudinal axis A₁ of the shaft 202, such that the end effector 204 is at a substantially zero angle relative to the shaft 202. Due to factors such as manufacturing tolerance and precision of measurement devices, the end effector 204 may not be at a precise zero angle relative to the shaft 202 in the unarticulated position, but nevertheless be considered “substantially aligned” thereto. In the articulated position, the longitudinal axes A₁, A₂ would be angularly offset from each other such that the end effector 204 is at a non-zero angle relative to the shaft 202.

In some embodiments, the surgical tool 200 may be supplied with electrical power (current) via a power cable 214 coupled to the housing 208. In other embodiments, the power cable 214 may be omitted and electrical power may be supplied to the surgical tool 200 via an internal power source, such as one or more batteries, capacitors, or fuel cells. In such embodiments, the surgical tool 200 may alternatively be characterized and otherwise referred to as an “electrosurgical instrument” capable of providing electrical energy to the end effector 204.

The power cable 214 may place the surgical tool 200 in electrical communication with a generator that supplies energy, such as electrical energy (e.g., radio frequency energy), ultrasonic energy, microwave energy, heat energy, or any combination thereof, to the surgical tool 200 and, more particularly, to the end effector 204. Accordingly, the generator may comprise a radio frequency (RF) source, an ultrasonic source, a direct current source, and/or any other suitable type of electrical energy source that may be activated independently or simultaneously.

The surgical tool 200 may be configured for bipolar or monopolar operation. In bipolar operation, the power cable 214 will include a supply conductor and a return conductor. Current can be supplied from a generator (not shown) to the blades 210, 212 via the supply conductor, and current can flow back to the generator via a return conductor. In applications where the surgical tool 200 is configured for monopolar operation, the electrical energy may comprise radio frequency (“RF”) energy exhibiting a frequency between about 100 kHz and 1 MHz. Low frequency RF energy causes ionic agitation or friction, in effect resistive heating, thereby increasing the temperature of target tissue. The electrical energy supplied to the end effector 204 is converted to heat and transferred to adjacent tissue to cut, cauterize, and/or coagulate the tissue (dependent upon the localized heating of the tissue), and thus may be particularly useful for sealing blood vessels or diffusing bleeding. Electrical energy (current) is then returned (dissipated) from the tissue through a return electrode, which typically comprises a grounding pad separately located on a patient's body.

The surgical tool 200 including monopolar blades 210, 212 is a highly-utilized tool for many procedures, and the life of such tools can be limited by the degradation of the cutting edge of the blades 210, 212 due to the monopolar energy employed during procedures. In conventional surgical tools, the blades 210, 212 are designed to be replaced at a hospital or at a service center to increase the procedure life of the surgical tool. According to embodiments of the present disclosure, however, the end effector 204 may include a blade retention system that allows the blades 210, 212 to be manually and easily replaced at bedside by an operator or user (e.g., a surgeon, a nurse, bedside assist, etc.), which can drastically increase the life of the end effector 204 and the surgical tool 200.

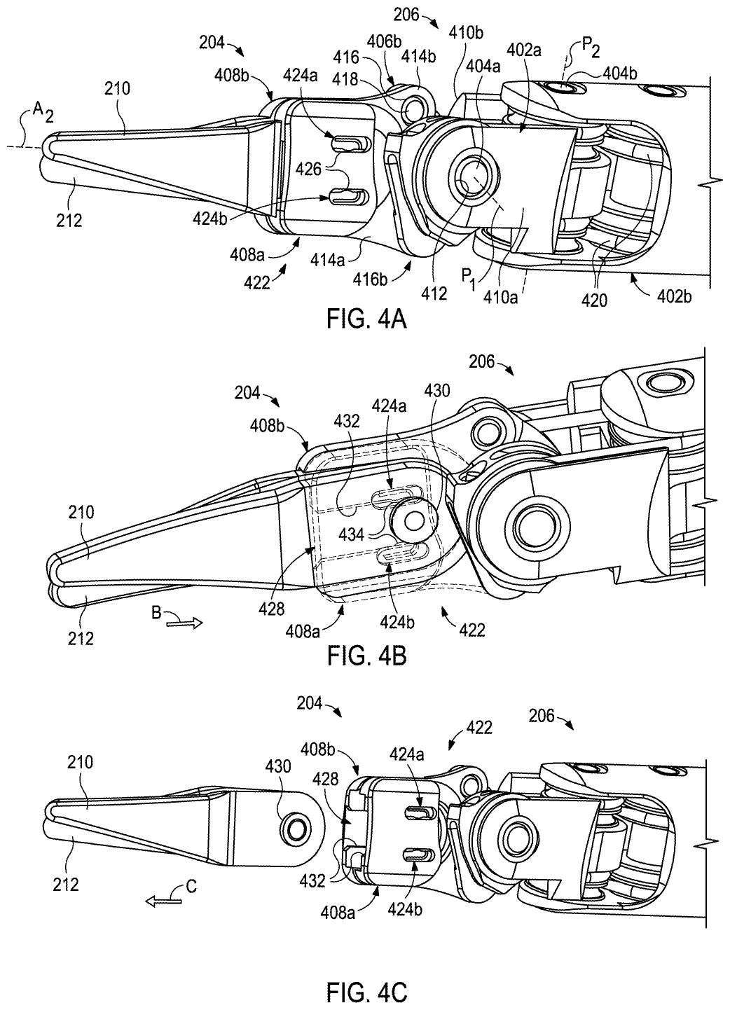

FIGS. 4A-4C are enlarged isometric views of the distal end of the surgical tool 200, according to one or more embodiments. More specifically, FIGS. 4A-4C depict enlarged views of the end effector 204 and the wrist 206, and further depict an example blade retention system 422 that secures the blades 210, 212 for operation. As described herein, the blade retention system 422 can be used to manually remove the blades 210, 212 and subsequently replace the used blades 210, 212 with a new set of blades, thus prolonging the useful life of the end effector 204.

Referring first to FIG. 4A, the wrist 206 is operatively coupled to the end effector 204 and includes a distal clevis 402a and a proximal clevis 402b. The end effector 204 (i.e., the blades 210, 212) is rotatably mounted to the distal clevis 402a at a first axle 404a, and the distal clevis 402a is rotatably mounted to the proximal clevis 402b at a second axle 404b. The wrist 206 provides a first pivot axis P₁ that extends through the first axle 404a and a second pivot axis P₂ that extends through the second axle 404b. The first pivot axis P₁ is substantially perpendicular (orthogonal) to the longitudinal axis A₂ of the end effector 204, and the second pivot axis P₂ is substantially perpendicular (orthogonal) to both the longitudinal axis A₂ and the first pivot axis P₁. Movement about the first pivot axis P₁ provides “pitch” articulation of the end effector 204, and movement about the second pivot axis P₂ provides “yaw” articulation of the end effector 204.

In the illustrated embodiment, the blades 210, 212 are actuatable between closed and open positions by movement about the first pivot axis P₁. More specifically, the end effector 204 includes first and second blade pulleys 406a and 406b (406b is mostly occluded), and first and second blade holders 408a and 408b mounted to the blade pulleys 406a,b, respectively. The first blade 210 is releasably secured to the first blade holder 408a, and the second blade 212 is releasably secured to the second blade holder 408b such that rotational movement of the first and second blade pulleys 406a,b correspondingly moves the blade holders 408a,b in the same direction, which moves the blades 210, 212 between the closed and open positions.

As illustrated, the distal clevis 402a includes a pair of distally extending arms 410a and 410b, and the first axle 404a is mounted to corresponding apertures 412 (only one visible) defined in each arm 410a,b. The blade pulleys 406a,b are mounted to the first axle 404a and rotatable about the first pivot axis P₁. As illustrated, each blade holder 408a,b provides or otherwise defines a proximally extending flange 414a and 414b, respectively, and each flange 414a,b may be mounted to the corresponding (adjacent) blade pulley 406a,b, respectively. In the illustrated embodiment, for example, each flange 414a,b defines a flange aperture 416 (only one shown), and the corresponding blade pulley 406a,b provides a pulley pin 418 (only one shown) configured to be received within the flange aperture 416. Mounting the blade holder 408a,b to the corresponding blade pulley 406a,b allows the blade holder 408a,b to rotate as the associated blade pulley 406a,b rotates.

While FIG. 4A shows the aperture 416 being provided by the flanges 414a,b, and the pins 418 being provided by the blade pulleys 406a,b, in other embodiments the flanges 414a,b may instead provide a pin and the blade pulleys 406a,b may alternatively provide an aperture that receives the pin, without departing from the scope of the disclosure.

A plurality of drive cables 420 pass through the wrist 206 and terminate at the blade pulleys 406a,b. The drive cables 420 form part of the cable driven motion system housed within the drive housing 208 (FIG. 2), and may comprise cables, bands, lines, cords, wires, woven wires, ropes, strings, twisted strings, elongate members, belts, shafts, flexible shafts, drive rods, or any combination thereof. The drive cables 420 can be made from a variety of materials including, but not limited to, a metal (e.g., tungsten, stainless steel, nitinol, etc.), a polymer (e.g., ultra-high molecular weight polyethylene), a synthetic fiber (e.g., KEVLAR®, VECTRAN®, etc.), an elastomer, or any combination thereof.

The drive cables 420 extend proximally from the end effector 204 to the drive housing 208 (FIG. 2) where they are operatively coupled to various actuation mechanisms or devices housed (contained) therein to facilitate longitudinal movement (translation) of the drive cables 420 within the lumen 410. Selective actuation of all or a portion of the drive cables 420 causes the wrist 206 to articulate and/or causes the blades 210, 212 to open or close. While only two drive cables 420 are visible in FIG. 4A, the surgical tool 200 may include more than two (e.g., four).

In particular, a pair of drive cables 420 may terminate at each blade pulley 406a,b and may be configured to “antagonistically” operate the corresponding blade 210, 212. Accordingly, the drive cables 420 may be characterized or otherwise referred to as “antagonistic” cables that cooperatively (yet antagonistically) operate to cause relative or tandem movement of the blade holders 408a,b and, therefore, relative or tandem movement of the blades 210, 212.

The end effector 204 may further include a blade retention system 422 that secures the blades 210, 212 for operation, but also allows a user to manually remove the blades 210, 212 and replace the old blades 210, 212 with a new set of blades quickly and efficiently. The blade holders 408a,b form part of the blade retention system 422 and each blade holder 408a,b may include or provide one or more retention mechanisms configured to releasably secure the blades 210, 212. In the illustrated embodiment, for example, each blade holder 408a,b may include a first or “upper” retention mechanism 424a and a second or “lower” retention mechanism 424b. In some embodiments, as illustrated, each blade holder 408a,b may further define upper and lower apertures 426 through which the upper and lower retention mechanisms 424a,b may be visible. In other embodiments, however, the apertures 426 may be omitted. The retention mechanisms 424a,b provided on each blade holder 408a,b may be configured to removably secure the associated blade 210, 212 within the interior of the corresponding blade holder 408a,b.

Referring now to FIGS. 4B and 4C, the blades 210, 212 are depicted in a first or “secured” state (FIG. 4B) and a second or “detached” state (FIG. 4C). In the secured state, the proximal end of each blade 210, 212 is received within an interior 428 of the blade holders 408a,b and removably secured to the corresponding blade holder 408a,b with the retention mechanisms 424a,b. As illustrated, the interior 428 may be cooperatively defined by the blade holders 408a,b. In particular, each blade holder 408a,b may independently define an interior or “pocket” that is sized to receive the proximal end of the corresponding blade 210, 212. In the detached state, the blades 210, 212 are removed from the interior 428 of the blade holders 408a,b.

The first blade holder 408a is shown in FIG. 4B in phantom (dashed lines) to enable viewing of the internal component parts of the blade retention system 422. The blade retention system 422 includes parts and functionality pertaining to both blade holders 408a,b and both blades 210, 212. The following discussion of the blade retention system 422 is related to the first blade holder 408a and the first blade 210, but is equally applicable to the second blade holder 408b and the second blade 212.

As illustrated, the first blade 210 provides a boss 430 extending laterally outward from a side surface of the blade 210 at or near a proximal end of the blade 210. In some embodiments, a longitudinal channel 432 may be defined within the interior 428 of the first blade holder 408a and sized to receive the boss 430 as the blade 210 is moved longitudinally into and out of the interior 428. In at least one embodiment, a height of the longitudinal channel 432 may be slightly larger than a diameter of the boss 430, which allows the boss 430 to be received within and slidably engage the longitudinal channel 432 as the blade 210 is moved (translated) proximally or distally within the interior 428.

The upper and lower retention mechanisms 424a,b may comprise flexible arms, extensions, or detents provided on or near the upper and lower walls of the longitudinal channel 432. In some embodiments, the retention mechanisms 424a,b may comprise axial extensions of the longitudinal channel 432 provided at or near its proximal end. The distal end of each retention mechanism 424a,b may be attached to the blade holder 408a at the upper and lower walls of the longitudinal channel 432, and the proximal end of each retention mechanism 424a,b may be free-floating, which allows the retention mechanisms 424a,b to flex laterally outward (i.e., up and down in FIG. 4B) upon engaging the boss 430.

As best seen in FIG. 4B, each retention mechanism 424a,b may provide and otherwise define an inner profile 434 configured to receive and engage the boss 430 when the blade 210 is advanced proximally into the longitudinal channel 432. In the illustrated embodiment, the inner profile 434 comprises a projection or protrusion that extends inward and otherwise into the longitudinal channel 432. The boss 430 engages the inner profile 434 as it advances proximally or distally within the longitudinal channel 432, and thereby causes the retention mechanisms 424a,b to flex laterally outward to accommodate the diameter of the boss 430. Once the boss 430 bypasses the inner profile 434 in the proximal direction, the retention mechanisms 424a,b may help secure the blade 210 within the interior 428.

To position the blade 210 in the secured state, as shown in FIG. 4B, the blade 210 may be advanced proximally into the interior 428 of the first blade holder 408a, as shown by the arrow B. As the blade 210 enters the interior 428, the boss 430 will locate and be received within the longitudinal channel 432, which will guide the boss 430 toward the retention mechanisms 424a,b. Once reaching the retention mechanisms 424a,b, the boss 430 will engage the inner profiles 434 and additional force (movement) in the proximal direction B which will cause the upper and lower retention mechanisms 424a,b to flex outward, thereby allowing the boss 430 to bypass the retention mechanisms 424a,b in the proximal direction B. Once the boss 430 bypasses the retention mechanisms 424a,b, the retention mechanisms 424a,b will spring back to their natural state and the inner profiles 434 will help secure the blade 210 within the blade holder 408a during operation.

To transition the blade 210 to the detached state, as shown in FIG. 4C, blade 210 may be moved in the distal direction, as shown by the arrow C. In some embodiments, this can be done by a user (e.g., surgeon, nurse, bedside assist, etc.) manually grasping onto the blade 210 and pulling the blade 210 distally C. Pulling the blade 210 in the distal direction C will drive the boss 430 against the retention mechanisms 424a,b and, more particularly, against the inner profiles 434. If sufficient force (movement) is applied in the distal direction C, the spring force of the retention mechanisms 424a,b will be overcome and the retention mechanisms 424a,b will flex outward, thereby allowing the boss 430 to bypass the retention mechanisms 424a,b. The blade 210 may then be advanced further distally C and out of the longitudinal channel 432 and the interior 428, thereby separating the blade 210 from the blade holder 408a.

Once the used (old) blades 210, 212 are removed from the corresponding blade holders 408a,b, a new set of blades (not shown) may then be removably secured to the blade holders 408a,b by repeating the above-described process of positioning the new blades in the secured state.

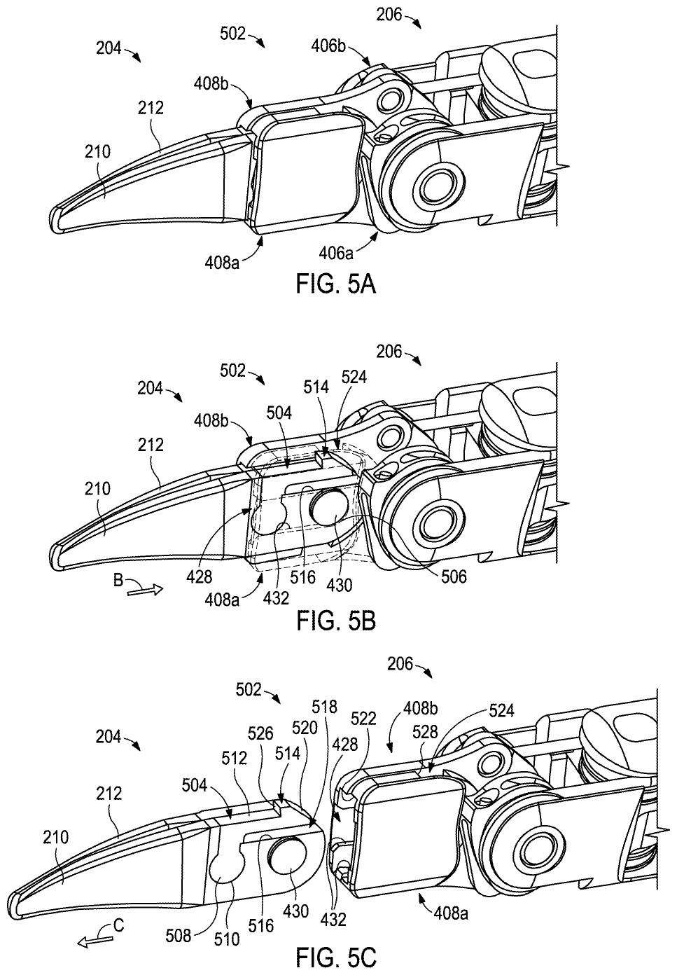

FIGS. 5A-5C are enlarged isometric views of the distal end of the surgical tool 200, according to one or more additional embodiments. More specifically, FIGS. 5A-5C depict enlarged views of the end effector 204 and the wrist 206, and further depict another example blade retention system 502 that secures the blades 210, 212 for operation but allows the blades 210, 212 to be manually removed and replaced, thus prolonging the useful life of the end effector 204.

Referring first to FIG. 5A, the wrist 206 may be the same as or substantially similar to the wrist 206 described with reference to FIG. 4A above, and therefore will not be described in detail. As illustrated, the blades 210, 212 are removably secured to the blade holders 408a,b, respectively, which are mounted to the blade pulleys 406a,b respectively, as generally described above. Rotational movement of the blade pulleys 406a,b will correspondingly move the blades 210, 212 between the closed and open positions, but can also move the blades 210, 212 in tandem in pitch.

Referring to FIGS. 5B and 5C, the blades 210, 212 are depicted in a first or “secured” state (FIG. 5B) and a second or “detached” state (FIG. 5C). In the secured state, the proximal end of each blade 210, 212 is received within the interior 428 of the blade holders 408a,b and removably secured to the corresponding blade holder 408a,b. In the detached state, the blades 210, 212 are removed distally from the interior 428 and the blade holders 408a,b. The blade holders 408a,b form part of the blade retention system 502.

The first blade holder 408a is shown in FIG. 5B in phantom (dashed lines) to enable viewing of various internal component parts of the blade retention system 502. The blade retention system 502 includes parts and functionality pertaining to both blade holders 408a,b and both blades 210, 212. The following discussion of the blade retention system 502 is related to the first blade holder 408a and the first blade 210, but is equally applicable to the second blade holder 408b and the second blade 212.

As illustrated, the first blade 210 provides the boss 430, and the longitudinal channel 432 is defined within the interior 428 of the first blade holder 408a to receive the boss 430 as the blade 210 is moved longitudinally into and out of the interior 428. In some embodiments, as best seen in FIG. 5B, the longitudinal channel 432 may terminate with a rounded (curved) end 506 configured to receive the boss 430 when the blade 210 is fully advanced into the interior 428.

The blade retention system 502 includes a retention mechanism 504 mounted to the blade 210. As best seen in FIG. 5C, the retention mechanism 504 includes a mounting feature 508 received within a mounting receptacle 510 defined in the body of the blade 210. Receiving the mounting feature 508 within the mounting receptacle 510 secures the retention mechanism 504 to the blade 210. In the illustrated embodiment, the mounting feature 508 comprises a bulbous projection or member, but could alternatively comprise any other feature capable of securing the retention mechanism 504 to the blade 210.

The retention mechanism 504 further includes a leg 512 extending longitudinally (proximally) from the mounting feature 508 and terminating in a latch feature 514. The blade 210 may define a longitudinal cutout or recess 516 sized to accommodate the leg 512. The recess 516 may be provided such that a gap 518 is defined between a top surface of the recess 516 and a bottom surface (underside) of the leg 512. The gap 518 may prove advantageous in allowing the leg 512 to flex into the recess 516 as the blade 210 is advanced into the interior 428.

The latch feature 514 may provide or define a slanted or angled proximal end 520. The angled proximal end 520 may be configured to engage an inner surface or wall 522 of the interior 428 as the blade 210 is advanced into the interior 428. Engaging the angled proximal end 520 on the inner wall 522 will cause the retention mechanism 504 (e.g., the leg 512) to flex into the gap 518, which allows the blade 210 to advance into the interior 428.

The latch feature 514 may be configured to locate and be received within a retention aperture 524 defined by the blade holder 408a at a proximal end of the inner wall 522. As illustrated, the latch feature 514 may further provide or define a shoulder 526 provided generally perpendicular to and otherwise extending from the leg 512. When the latch feature 514 is received within the retention aperture 524, the shoulder 526 may be configured to engage an end wall 528 of the retention aperture 524, thereby securing the blade 210 within the interior 428 and otherwise preventing the blade 210 from being removed from the interior 428.

While the retention mechanism 504 is depicted as being mounted to the blade 210, it is contemplated herein that the retention mechanism 504 may alternatively be mounted within the interior 428 of the blade holder 408a. In such embodiments, the blade 210 may include or define various features and structures configured to engage the retention mechanism 504, thereby preventing the blade 210 from being removed from the interior 428.

To position the blade 210 in the secured state, as shown in FIG. 5B, the blade 210 may be advanced proximally B into the interior 428 of the first blade holder 408a. As the blade 210 enters the interior 428, the boss 430 will locate and be received within the longitudinal channel 432, which will help guide the blade 210 into the interior 428. As the blade 210 is advanced into the interior 428, the angled proximal end 520 of the latch feature 514 will engage the inner wall 522 and cause the leg 512 to flex into the recess 516 (e.g., the gap 518). The blade 210 is then advanced proximally B until the latch feature 514 locates the retention aperture 524, at which point the leg 512 is able to flex outward to its natural state and the latch feature 514 is received within the retention aperture 524. Once the latch feature 514 is received within the retention aperture 524, the shoulder 526 will engage the end wall 528, thereby effectively securing the blade 210 within the blade holder 408a during operation. In some embodiments, the latch feature 514 may configured to be received within the retention aperture 524 at about the same time the boss 430 engages and is otherwise received by the rounded end 506 of the longitudinal channel 432. Structural engagement at both locations helps axially secure the blade 210 within the blade holder 408a.

To transition the blade 210 to the detached state, as shown in FIG. 5C, the latch feature 514 may first be disengaged from the retention aperture 524. In some embodiments, this can be done manually by advancing a tool (e.g., a rod, a screwdriver, or the like) into the retention aperture 524 and engaging the latch feature 514. The tool may be used to force (flex) the latch feature 514 until the shoulder 526 disengages from the end wall 528. Once the shoulder 526 is disengaged from the end wall 528, the blade 210 may then be translated in the distal direction C. In some embodiments, this can be done by a user (e.g., surgeon, nurse, bedside assist, etc.) manually grasping onto the blade 210 and pulling the blade 210 distally C. As the blade 210 moves distally C, the leg 512 is flexed inward and into the gap 518 until the latch feature 514 exits the interior 428. Once the latch feature 514 exits the interior 428, the leg 512 may be able to flex back outward to its natural state. The blade 210 is then at this point separated from the blade holder 408a.

Once the used (old) blades 210, 212 are removed from the corresponding blade holders 408a,b, a new set of blades (not shown) may then be removably secured to the blade holders 408a,b by repeating the above-described process of positioning the new blades in the secured state.

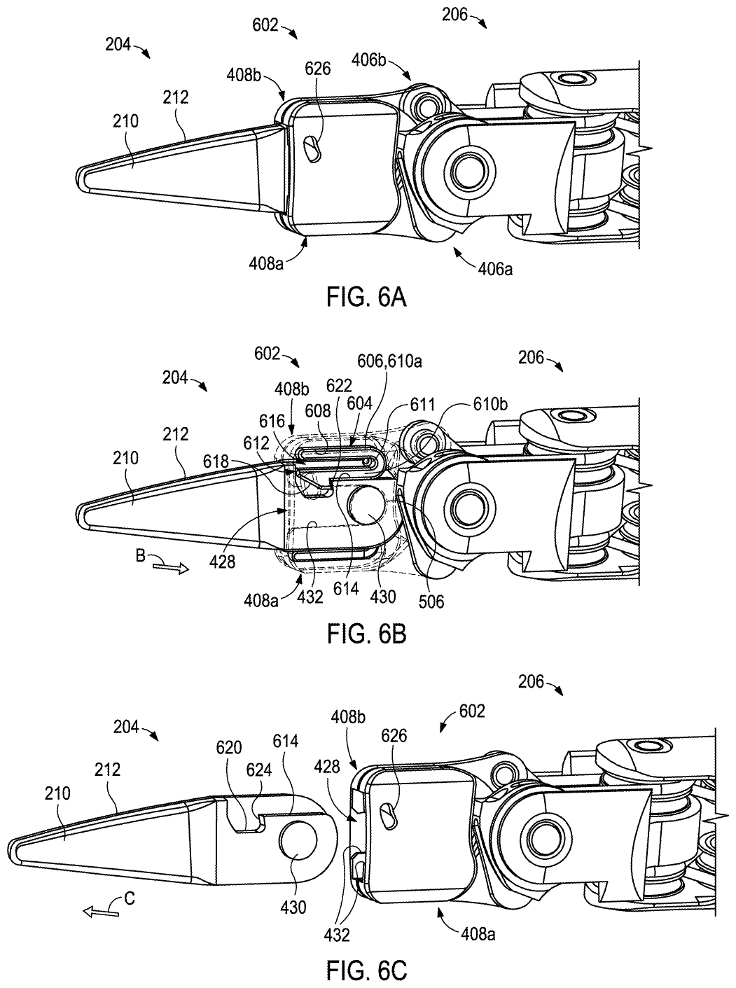

FIGS. 6A-6C are enlarged isometric views of the distal end of the surgical tool 200, according to one or more additional embodiments. More specifically, FIGS. 6A-6C depict enlarged views of the end effector 204 and the wrist 206, and further depict another example blade retention system 602 that secures the blades 210, 212 for operation and allows the blades 210, 212 to be manually removed and replaced, thus prolonging the useful life of the end effector 204.

Referring first to FIG. 6A, the wrist 206 may be the same as or substantially similar to the wrist 206 described with reference to FIG. 4A above, and therefore will not be described in detail. As illustrated, the blades 210, 212 are removably secured to the blade holders 408a,b, respectively, which are mounted to the blade pulleys 406a,b respectively, as generally described above. Rotational movement of the blade pulleys 406a,b will correspondingly move the blades 210, 212 between the closed and open positions, but can also move the blades 210, 212 in tandem in pitch.

Referring now to FIGS. 6B and 6C, the blades 210, 212 are depicted in a first or “secured” state (FIG. 6B) and a second or “detached” state (FIG. 6C). In the secured state, the proximal end of each blade 210, 212 is received within the interior 428 of the blade holders 408a,b and removably secured to the corresponding blade holder 408a,b. In the detached state, the blades 210, 212 are removed distally from the interior 428 and the blade holders 408a,b. The blade holders 408a,b form part of the blade retention system 602.

The blade holders 408a,b are shown in FIG. 6B in phantom (dashed lines) to enable viewing of various internal component parts of the blade retention system 602. The blade retention system 602 includes parts and functionality pertaining to both blade holders 408a,b and both blades 210, 212. The following discussion of the blade retention system 602 is related to the first blade holder 408a and the first blade 210, but is equally applicable to the second blade holder 408b and the second blade 212.

As illustrated, the first blade 210 provides the boss 430, and the longitudinal channel 432 is defined within the interior 428 of the first blade holder 408a to receive the boss 430 as the blade 210 is moved longitudinally into and out of the interior 428. As best seen in FIG. 6B, the longitudinal channel 432 may terminate with the rounded (curved) end 506 configured to receive the boss 430 when the blade 210 is fully advanced into the interior 428.

The blade retention system 602 includes a retention mechanism 604 mounted to the blade holder 408a and engageable with the blade 210 as the blade 210 is advanced into the interior 428. As best seen in FIG. 6B, the retention mechanism 604 includes a body 606 received within a mounting receptacle 608 defined in the body of the blade holder 408a. In some embodiments, as illustrated, the body 606 may comprise a generally U-shaped structure having opposing first and second (e.g., “outer” and “inner”) legs 610a and 610b. In at least one embodiment, the mounting receptacle 608 may also define a generally U-shaped channel or groove sized to receive the body 606. In other embodiments, however, the mounting receptacle 608 may exhibit any other shape suitable for receiving and securing the body 606 therein. In the illustrated embodiment, the mounting receptacle 608 includes a mounting pin 611 about which the U-shaped body 606 can extend when received within the mounting receptacle 608.

The inner leg 610b terminates in a latch feature 612, and the blade 210 may define a longitudinal cutout or recess 614 sized to accommodate the inner leg 610b. A gap 616 is defined between the outer and inner legs 610a,b, which may prove advantageous in allowing the inner leg 610b to flex as the blade 210 is advanced into the interior 428.

The latch feature 612 may provide or otherwise define a slanted or angled distal end 618. The angled distal end 618 may be configured to engage the proximal end of the recess 614 as the blade 210 is advanced into the interior 428. Engaging the angled distal end 618 on the recess 614 will cause the retention mechanism 604 (e.g., the inner leg 610b) to flex into the gap 616, which allows the blade 210 to advance into the interior 428.

The latch feature 612 may be configured to locate and be received within a retention receptacle 620 (best seen in FIG. 6C) defined on the blade 210 and contiguous with the recess 614. As illustrated, the latch feature 612 may further provide or otherwise define a shoulder 622 provided generally perpendicular to and otherwise extending from the inner leg 610b. When the latch feature 612 is received within the retention receptacle 620, the shoulder 620 may be configured to engage an end wall 624 (best seen in FIG. 6C) of the retention receptacle 620, thereby securing the blade 210 within the interior 428 and otherwise preventing the blade 210 from being removed from the interior 428.

To position the blade 210 in the secured state, as shown in FIG. 6B, the blade 210 may be advanced proximally B into the interior 428 of the first blade holder 408a. As the blade 210 enters the interior 428, the boss 430 will locate and be received within the longitudinal channel 432, which will help guide the blade 210 into the interior 428. As the blade 210 is advanced into the interior 428, the angled proximal end 618 of the latch feature 612 will engage the proximal end of the recess 614, which will cause the inner leg 610b to flex into the gap 616. The blade 210 is then advanced proximally B until the latch feature 612 locates the retention receptacle 620, at which point the inner leg 610b is able to flex back to its natural state and the latch feature 612 is received within the retention receptacle 620. Once the latch feature 612 is received within the retention receptacle 620, the shoulder 620 will engage the end wall 624, thereby effectively securing the blade 210 within the blade holder 408a during operation. In some embodiments, the latch feature 612 may configured to be received within the retention receptacle 620 at about the same time the boss 430 engages and is otherwise received by the rounded end 506 of the longitudinal channel 432. Structural engagement at both locations helps axially secure the blade 210 within the blade holder 408a.

To transition the blade 210 to the detached state, as shown in FIG. 6C, the latch feature 612 may first be disengaged from the retention receptacle 620. This can be done manually by advancing a tool (e.g., a rod, a screwdriver, or the like) into an aperture 626 (FIGS. 6A and 6C) defined in the sidewall of the blade holder 408a. The aperture 626 may be aligned with the latch feature 612 and, more particularly, aligned with the location where the latch feature 612 is received within the retention receptacle 620. Advancing the tool into the aperture 626 may engage the angled distal end 618 of the latch feature 612, and thereby force (flex, move) the latch feature 612 and the inner leg 610b until the shoulder 620 disengages from the end wall 624. Once the shoulder 620 is disengaged from the end wall 624, the blade 210 may then be translated distally C. In some embodiments, this can be done by a user (e.g., surgeon, nurse, bedside assist, etc.) manually grasping onto the blade 210 and pulling the blade 210 distally C. As the blade 210 moves distally C, the inner leg 610b remains flexed into the gap 616 until the latch feature 612 exits the recess 614. Once the blade 210 advances distally C sufficient to allow the latch feature 612 to exit the recess 614, the inner leg 610b may flex back to its natural state. The blade 210 is then at this point separated from the blade holder 408a.

Once the used (old) blades 210, 212 are removed from the corresponding blade holders 408a,b, a new set of blades (not shown) may then be removably secured to the blade holders 408a,b by repeating the above-described process of positioning the new blades in the secured state.

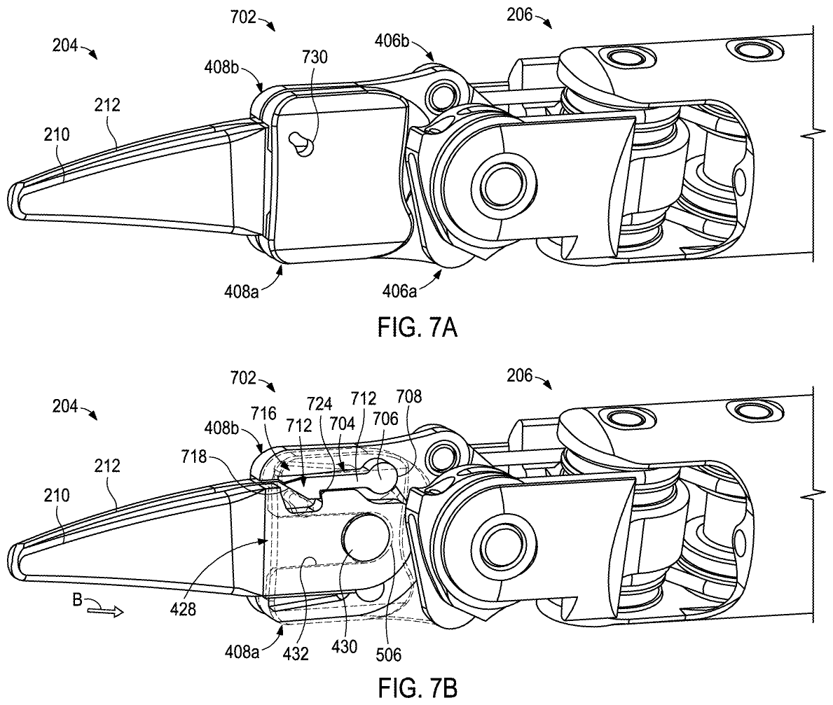

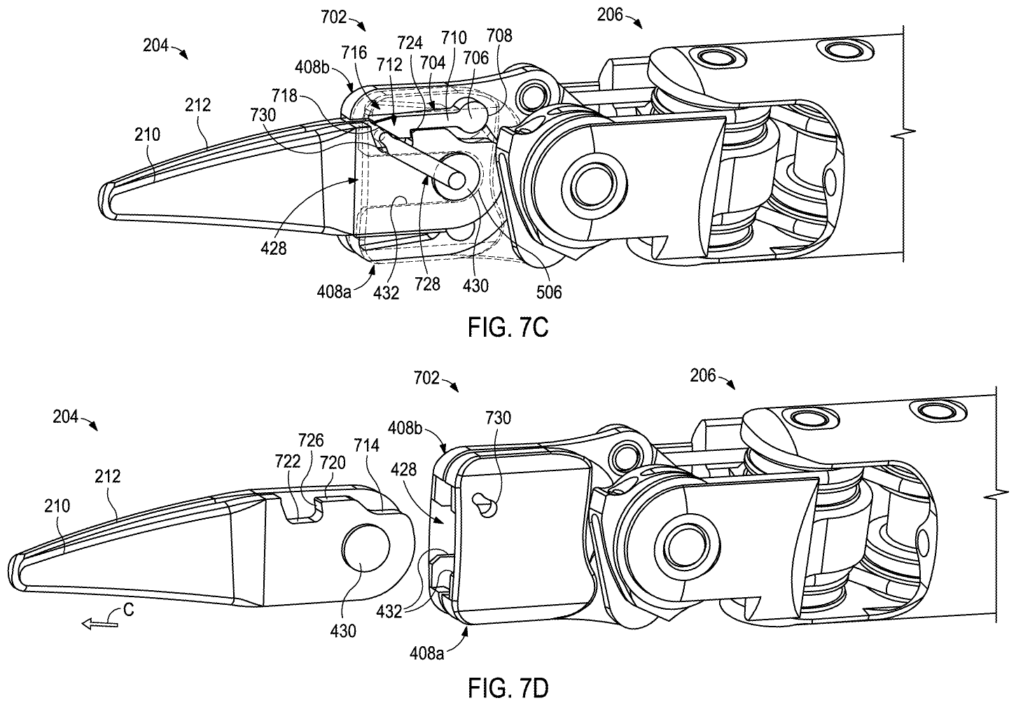

FIGS. 7A-7D are enlarged isometric views of the distal end of the surgical tool 200, according to one or more additional embodiments. More specifically, FIGS. 7A-7D depict enlarged views of the end effector 204 and the wrist 206, and further depict another example blade retention system 702 that secures the blades 210, 212 for operation and allows the blades 210, 212 to be manually removed and replaced, thus prolonging the useful life of the end effector 204.

Referring first to FIG. 7A, the wrist 206 may be the same as or substantially similar to the wrist 206 described with reference to FIG. 4A above, and therefore will not be described in detail. As illustrated, the blades 210, 212 are removably secured to the blade holders 408a,b, respectively, which are mounted to the blade pulleys 406a,b respectively, as generally described above. Rotational movement of the blade pulleys 406a,b will correspondingly move the blades 210, 212 between the closed and open positions, but can also move the blades 210, 212 in tandem in pitch.

Referring to FIGS. 7B-7D, the blades 210, 212 are depicted in a first or “secured” state (FIGS. 7B and 7C) and a second or “detached” state (FIG. 7D). In the secured state, the proximal end of each blade 210, 212 is received within the interior 428 of the blade holders 408a,b and removably secured to the corresponding blade holder 408a,b. In the detached state, the blades 210, 212 are removed distally from the interior 428 and the blade holders 408a,b. The blade holders 408a,b form part of the blade retention system 702.

The first blade holder 408a is shown in FIGS. 7B and 7C in phantom (dashed lines) to enable viewing of various internal component parts of the blade retention system 702. The blade retention system 702 includes parts and functionality pertaining to both blade holders 408a,b and both blades 210, 212. The following discussion of the blade retention system 702, however, is related to the first blade holder 408a and the first blade 210, but is equally applicable to the second blade holder 408b and the second blade 212.

As illustrated, the first blade 210 provides the boss 430, and the longitudinal channel 432 is defined within the interior 428 of the first blade holder 408a to receive the boss 430 as the blade 210 is moved longitudinally into and out of the interior 428. As best seen in FIGS. 7B and 7C, the longitudinal channel 432 may terminate with the rounded (curved) end 506 configured to receive the boss 430 when the blade 210 is fully advanced into the interior 428.

The blade retention system 702 includes a retention mechanism 704 (FIGS. 7B and 7C) mounted to the blade holder 408a and engageable with the blade 210 as the blade 210 is advanced into the interior 428. The retention mechanism 704 includes a mounting feature 706 received within a mounting receptacle 708 defined in the body of the blade holder 408a, and receiving the mounting feature 706 within the mounting receptacle 708 secures the retention mechanism 704 to the blade holder 408a. In the illustrated embodiment, the mounting feature 706 comprises a bulbous projection or member, but could alternatively comprise any other feature capable of securing the retention mechanism 704 to the blade holder 408a.

The retention mechanism 704 further includes a leg 710 extending longitudinally (distally) from the mounting feature 706 and terminating in a latch feature 712. The blade 210 may define a longitudinal cutout or recess 714 (FIG. 7D) sized to accommodate the leg 710. A gap 716 is defined in the blade holder 408a adjacent (e.g., above) the leg 710, which may prove advantageous in allowing the leg 710 to flex into the gap 716 as the blade 210 is advanced into the interior 428.

The latch feature 712 may provide or otherwise define a slanted or angled distal end 718 configured to engage the recess 714 as the blade 210 is advanced into the interior 428. In some embodiments, as best seen in FIG. 7D, the recess 714 may provide or define a projection 720 along the longitudinal length of the recess 714. Engaging the angled distal end 718 on the recess 714 and/or the projection 720 will cause the retention mechanism 704 (e.g., the leg 710) to flex into the gap 716, which allows the blade 210 to advance into the interior 428.

The latch feature 712 may be configured to locate and be received within a retention receptacle 722 (best seen in FIG. 7D) defined on the blade 210 and contiguous with the recess 714. As illustrated, the latch feature 712 may further provide or otherwise define a shoulder 724 provided generally perpendicular to and otherwise extending from the leg 710. When the latch feature 712 is received within the retention receptacle 722, the shoulder 724 may be configured to engage an end wall 726 (FIG. 7D) of the retention receptacle 722, thereby securing the blade 210 within the interior 428 and otherwise preventing the blade 210 from being removed from the interior 428.

To position the blade 210 in the secured state, as shown in FIGS. 7B-7C, the blade 210 may be advanced proximally B into the interior 428 of the first blade holder 408a. As the blade 210 enters the interior 428, the boss 430 will locate and be received within the longitudinal channel 432, which will help guide the blade 210 into the interior 428. As the blade 210 is advanced into the interior 428, the angled proximal end 718 of the latch feature 712 will engage the recess 714, and advancing the blade 210 further into the interior 428 will force the angled proximal end 714 into engagement with the projection 720. This causes the leg 710 to flex into the gap 716. In some embodiments, as illustrated, the proximal end of the projection 720 may be angled and configured to slidably engage the angled proximal end 718 of the latch feature 712. The blade 210 is then further advanced proximally B until the latch feature 712 locates the retention receptacle 722, at which point the leg 710 is able to flex outward to its natural state and the latch feature 712 is received within the retention receptacle 722. Once the latch feature 712 is received within the retention receptacle 722, the shoulder 724 will engage the end wall 726, thereby helping to secure the blade 210 within the blade holder 408a during operation. In some embodiments, the latch feature 712 may configured to be received within the retention receptacle 722 at about the same time the boss 430 engages and is otherwise received by the rounded end 506 of the longitudinal channel 432. Structural engagement at both locations helps axially secure the blade 210 within the blade holder 408a.

To transition the blade 210 to the detached state, as shown in FIG. 7D, the latch feature 712 may first be disengaged from the retention receptacle 722. This can be done manually by advancing a tool 728 (FIG. 7C), such as a rod, a screwdriver, or the like, into an aperture 730 defined in the blade holder 408a. The tool 728 may be the same as or similar to the tools mentioned above with reference to the prior embodiments. The aperture 730 may be aligned with the latch feature 712 and, more particularly, aligned with the location where the latch feature 712 is received within the retention receptacle 722. Advancing the tool 730 into the aperture 730 may engage the angled distal end 718 of the latch feature 712 and flex the leg 710 into the gap 716 until the shoulder 724 disengages from the end wall 726. Once the shoulder 724 is disengaged from the end wall 726, the blade 210 may then be translated distally C. In some embodiments, this can be done by a user (e.g., surgeon, nurse, bedside assist, etc.) manually grasping onto the blade 210 and pulling the blade 210 distally C. As the blade 210 moves distally C, the leg 710 remains flexed into the gap 716 until the latch feature 712 bypasses the projection 720. Once the blade 210 advances distally C and the latch feature 714 bypasses the projection 720, the leg 710 may be able to flex back to its natural state. The blade 210 is then at this point separated from the blade holder 408a.

Once the used (old) blades 210, 212 are removed from the corresponding blade holders 408a,b, a new set of blades (not shown) may then be removably secured to the blade holders 408a,b by repeating the above-described process of positioning the new blades in the secured state.

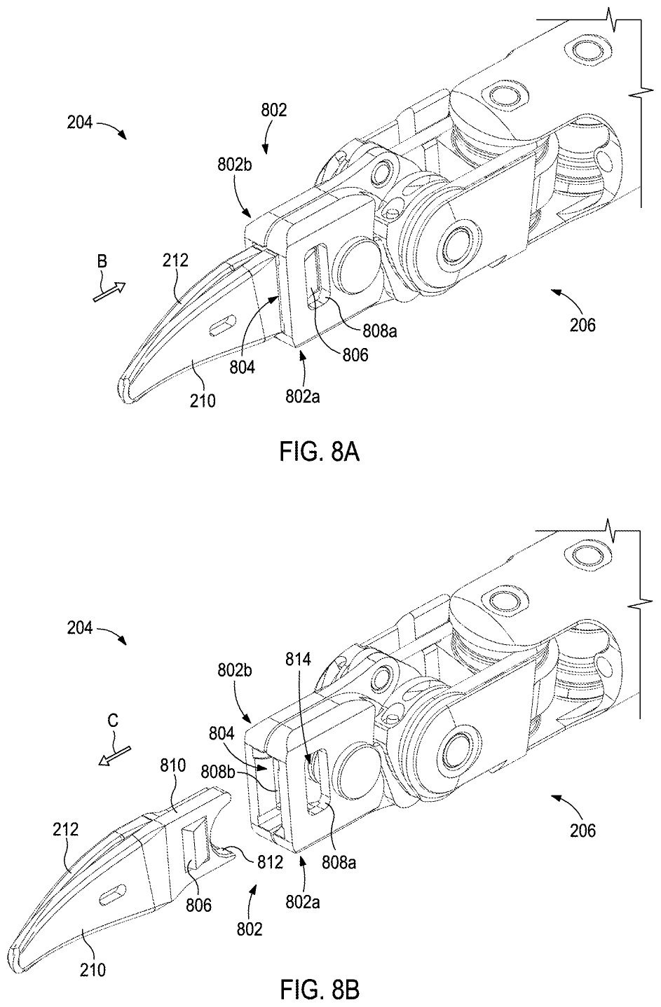

FIGS. 8A and 8B are enlarged isometric views of the distal end of the surgical tool 200, according to one or more additional embodiments. More specifically, FIGS. 8A-8B depict enlarged views of the end effector 204 and the wrist 206, and further depict another example blade retention system 802 that secures the blades 210, 212 for operation and allows the blades 210, 212 to be manually removed and replaced, thus prolonging the useful life of the end effector 204.

The wrist 206 may be the same as or substantially similar to the wrist 206 described with reference to FIG. 4A above, and therefore will not be described in detail. As illustrated, the blades 210, 212 are removably secured to first and second blade holders 802a and 802b, respectively, which are mounted to the blade pulleys 406a,b respectively, as generally described above. Rotational movement of the blade pulleys 406a,b will correspondingly move the blades 210, 212 between the closed and open positions, but can also move the blades 210, 212 in tandem in pitch.

The blades 210, 212 are depicted in FIG. 8A in a first or “secured” state, and depicted in FIG. 8B in a second or “detached” state. In the secured state, the proximal end of each blade 210, 212 is received within an interior 804 of the blade holders 802a,b and removably secured to the corresponding blade holder 802a,b. In the detached state, the blades 210, 212 are removed distally from the interior 804 and the blade holders 802a,b. The blade holders 802a,b form part of the blade retention system 802.

The blade retention system 802 includes a retention mechanism 806 (only one visible) provided on and otherwise defined by each blade 210, 212 and receivable within corresponding retention apertures 808a and 808b defined on the sidewall of each blade holder 802a,b. As best seen in FIG. 8B, the retention mechanism 806 comprises a leaf spring or the like provided on the shank 810 (e.g., proximal end) of each blade 210, 212 (only one retention mechanism 806 visible in FIG. 8B). Receiving the retention mechanism 806 within the corresponding retention aperture 808a,b secures the associated blade 210, 212 to the adjacent blade holder 802a,b and otherwise prevents the blades 210, 212 from being removed from the interior 804.

To position the blades 210, 212 in the secured state, as shown in FIG. 8A, the blades 210, 212 may be advanced proximally B into the interior 804 of the blade holders 802a,b. As the blades 210, 212 are advanced into the interior 804, the retention mechanism 806 provided on each blade 210, 212 will engage the opening to the interior 804, which will cause the retention mechanism 806 to flex laterally inward to allow the blades 210, 212 to enter the interior 804. The blades 210, 212 are then further advanced proximally B until the retention mechanisms 806 locate the corresponding retention aperture 808a,b at which point the retention mechanisms 806 are able to spring outward to their natural state and be received within the retention aperture 808a,b. Once the retention mechanisms 806 are received within the corresponding retention aperture 808a,b, the blades 210, 212 will be effectively secured within the blade holders 802a,b during operation.

Referring to FIG. 8B, the shank 810 of each blade 210, 212 may define an arcuate end 812, and a boss 814 may be provided by each blade holder 802a,b within the interior 804. In some embodiments, the blades 210, 212 may be advanced proximally B into the interior 804 until the arcuate ends 812 engage the corresponding bosses 814. In at least one embodiment, the retention mechanisms 806 may be received within the corresponding retention aperture 808a,b at about the same time the arcuate ends 812 engage the corresponding bosses 814. Structural engagement at both locations helps axially secure the blades 210, 212 within the blade holders 802a,b.

To transition the blades 210, 212 to the detached state, as shown in FIG. 8B, the retention mechanisms 806 may first be disengaged from the corresponding retention aperture 808a,b. In some embodiments, this can be done manually by advancing a tool (e.g., the tool 728 of FIG. 7C) into the retention apertures 808a,b and forcing (flexing) the retention mechanisms 806 out of engagement with the retention apertures 808a,b. Once the retention mechanisms 806 are disengaged from the corresponding retention apertures 808a,b, the blades 210, 212 may then be translated distally C. In some embodiments, this can be done by a user (e.g., surgeon, nurse, bedside assist, etc.) manually grasping onto the blades 210, 212 and pulling the blades 210, 212 distally C. As the blades 210, 212 move distally C, the retention mechanisms 806 remain flexed inward until exiting the interior 804 of the blade holders 802a,b. The blades 210, 212 are then at this point separated from the blade holders 802a,b.

In other embodiments, however, and as described in more detail below, a specially designed blade installation and removal tool may be provided to transition the blades 210, 212 between the secured and attached states. In such embodiments, the blade installation and removal tool may be provided to the user (e.g., surgeon, nurse, bedside assist, etc.) along with a new set of blades, and the user may be able to use the blade installation and removal tool to remove the old blades and subsequently install new blades. Moreover, the blade installation and removal tool may be made of rigid but inexpensive materials and, therefore, may be single-use and disposable.

FIG. 9 is an isometric, exploded view of the blades 210, 212 of FIGS. 8A-8B, according to one or more embodiments. As illustrated, each blade 210, 212 provides a blade body 902 having a first or “distal” end 904a and a second or “proximal” end 904b opposite the distal end 904a. In one or more embodiments, the blades 210, 212 may be made of stainless steel, titanium, or another rigid and conductive material. Moreover, the blades 210, 212 may be may be manufactured via stamping, but could alternatively be machined.

The blade body 902 includes the shank 810, which is located at or near the proximal end 904b. As discussed above, the retention mechanisms 806 of each blade 210, 212 may be provided on the shank 810, but could alternatively be provided at other locations. As illustrated, each retention mechanism 806 comprises a leaf spring or the like formed into the blade body 902, and is configured to extend laterally outward in its natural state. The arcuate end 812 may be defined and otherwise provided at or near the proximal end 904b.

According to embodiments of the present disclosure, a fixture aperture 906 may be defined in the blade body 902 of each blade 210, 212 near (closer to) the distal end 904a. As described in more detail below, a blade installation and removal tool may be configured to attach to (grasp onto) the blades 210, 212 at the fixture apertures 906, thereby allowing a user to manually install or remove the blades 210, 212. In the illustrated embodiment, each blade 210, 212 includes a single aperture 906, but it is contemplated herein to include multiple apertures in one or both of the blades to help in the installation and removal processes.

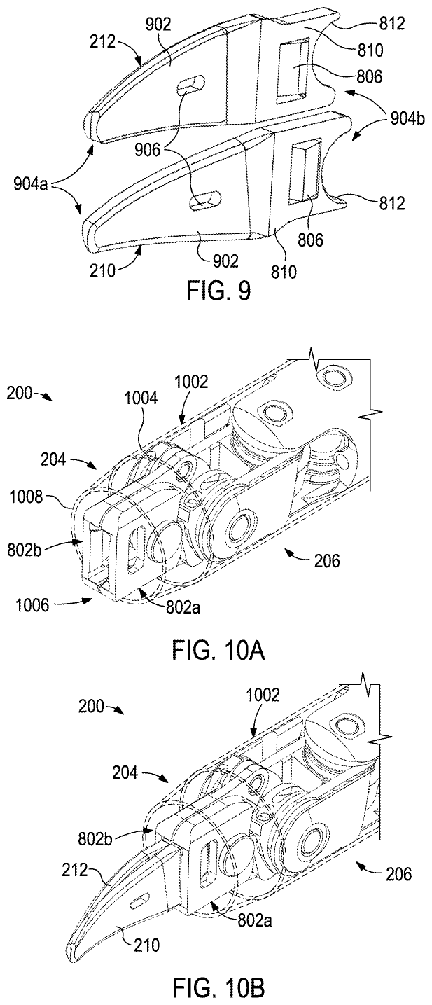

FIGS. 10A and 10B are isometric and end views of the surgical tool showing before and after installation states of the blades 210, 212, respectively, according to one or more embodiments. More specifically, FIG. 10A shows the end effector 204 without the blades 210, 212, and FIG. 10B depicts the blades 210, 212 secured to the blade holders 802a,b, as generally described above.

In the illustrated embodiment, the surgical tool 200 may further include a protective sleeve 1002 configured to insulate various live (energized) portions of the end effector 204 and the wrist 206, and thereby protect the patient from stray electrical discharge during operation. The sleeve 1002 may comprise an elongate and generally cylindrical body 1004. The body 1004 may be sized to extend over portions of the end effector 204 and the wrist 206, and when the sleeve 1002 is properly positioned for use, the blades 210, 212 protrude out a distal end 1006 of the body 1104 where an aperture 1008 is defined. When the sleeve 1002 is properly positioned (installed), electrical current can only be conducted to patient tissue as intended at the exposed blades 210, 212. Advantageously, since the blades 210, 212 are able to be manually installed at bedside, as generally described above with reference to FIGS. 8A-8B, the protective sleeve 1002 may first be installed over the end effector 204 and the wrist 206. The blades 210, 212 may then be attached to the blade holders 802a,b, thereby placing the end effector 204 in an operational and ready position.

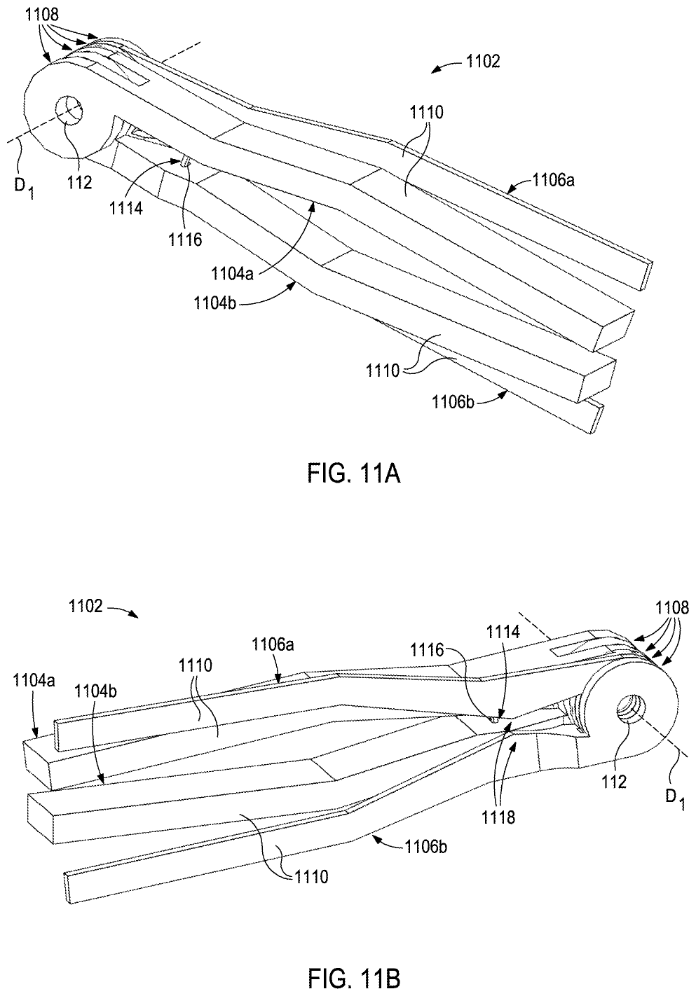

FIGS. 11A and 11B are left and right isometric views, respectively, of an example blade installation and removal tool 1102, according to one or more embodiments of the present disclosure. The blade installation and removal tool 1102 (hereafter “the tool 1102”) may be used to install and uninstall blades (e.g., the blades 210, 212) on an end effector (e.g., the end effector 204).

As illustrated, the tool 1102 includes opposing first and second securing handles 1104a and 1104b, and opposing first and second release handles 1106a and 1106b. The handles 1104a,b and 1106a,b may be made of a rigid material and, one or more embodiments, may further be made of an inexpensive material such as, but not limited to, a plastic, a metal (e.g., aluminum), a composite material, etc. In such embodiments, the tool 1102 may be designed for single-use and may be readily disposable after installing or uninstalling the blades 210, 212.

Each handle 1104a,b and 1106a,b includes a head 1108 and a handle portion 1110 extending from the head 1108. The heads 1108 are rotatably or pivotably mounted to a common axle 1112 and pivotable about a common pivot axis D₁. In example operation, as described below, a user (e.g., surgeon, nurse, bedside assist, etc.) may be able to grasp one or more of the handle portions 1110 and manipulate the angular position of the corresponding handles 1104a,b and 1106a,b by rotating the handle portions 1110 about the common pivot axis D₁.

In some embodiments, as illustrated, one or more the heads 1108 may exhibit a generally circular cross-section. In other embodiments, however, one or more of the heads 1108 may exhibit a different cross-sectional shapes including, but not limited to, polygonal, oval, ovoid, or any combination thereof. Moreover, in some embodiments, one or more of the heads 1108 may include two head portions laterally offset from each other, where the head 1108 of one handle 1104a,b and 1106a,b is received between the two head portions of an adjacent handle 1104a,b and 1106a,b. In the illustrated embodiment, for example, the head 1108 of the first securing handle 1104a includes two head portions laterally offset from each other, and the head 1108 of the second securing handle 1104b is received between and otherwise interposes the two head portions of the first securing handle 1104a. Similarly, the head 1108 of the second release handle 1106b includes two head portions, and the head 1108 of the first release handle 1106a is received between and otherwise interposes the two head portions of the second release handle 1106b.

The tool 1102 may further include a boss feature or “fixture projection” 1114 extending from the inner surface of one or both of the securing handles 1104a,b; e.g., extending from the handle portion 1110 of one or both of the securing handles 1104a,b. The fixture projection 1114 may be configured to extend through and be received within the coaxially aligned fixture apertures 906 (FIG. 9) defined in each blade 210, 212. Receiving the fixture projection 1114 within the fixture apertures 906 will effectively secure the blades 210, 212 to the tool 1102, which may help the user manipulate the blades 210, 212 to be received at the end effector 204.

In the illustrated embodiment, the fixture projection 1114 extends from the inner surface of the handle portion 1110 of the first securing handle 1104a, but in other embodiments the fixture projection 1114 may comprise two opposing portions extend toward each other from the inner surfaces of each of the securing handles 1104a,b. In some embodiments, the fixture projection 1114 may extend from the inner surface of the handle portion 1110 of the first securing handle 1104a and configured to engage the inner surface of the handle portion 1110 of the second securing handle 1104b when the securing handles 1104a,b are rotated toward each other and to a closed position. In other embodiments, however, the inner surface of the handle portion 1110 of the second securing handle 1104b may define a fixture aperture 1116 sized to receive the fixture projection 1114. In such embodiments, when the securing handles 1104a,b are rotated toward each other to the closed position, the fixture projection 1114 may align with and be received within the fixture aperture 1116.

As best seen in FIG. 11B, the release handles 1106a,b may each define opposing release projections 1118 provided on the inner surface of the handle portion 1110 of each release handle 1106a,b. When the release handles 1106a,b are rotated toward each other and to a closed position, the release projections 1118 may be configured to align with and extend into the retention apertures 808a,b (FIGS. 8A-8B) to engage the adjacent retention mechanisms 806 (FIGS. 8A-8B). In some embodiments, as illustrated, the release projections 1118 may exhibit an angled or raised point. In other embodiments, however the release projections 1118 may be curved, arcuate, rounded, flat (e.g., plateau), or any combination thereof, as long as they are able to engage and depress the retention mechanisms 806.

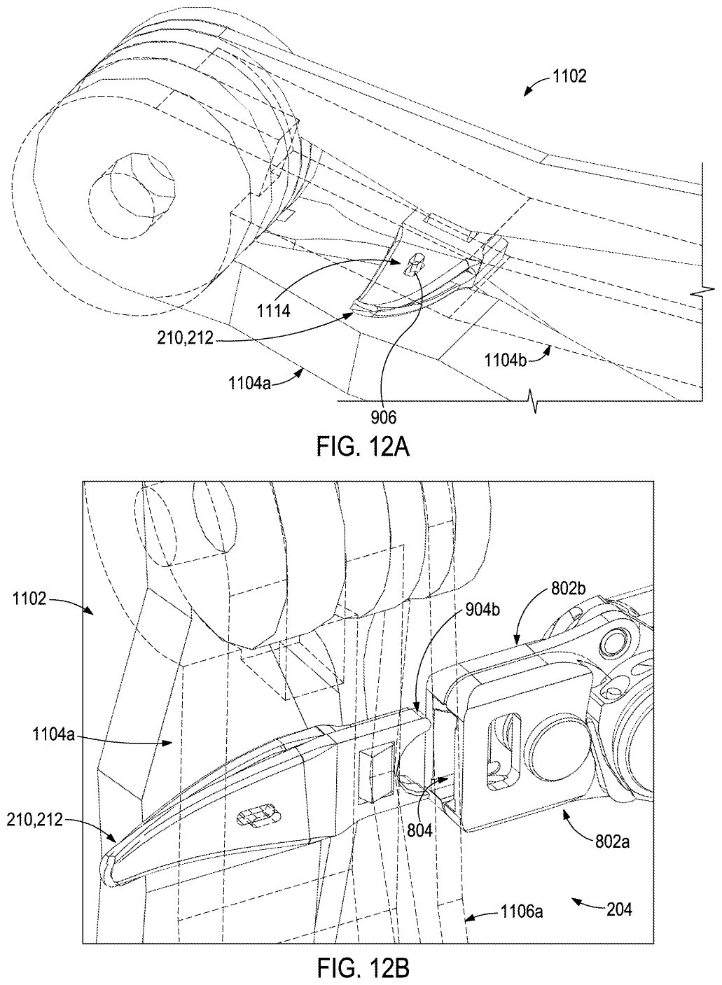

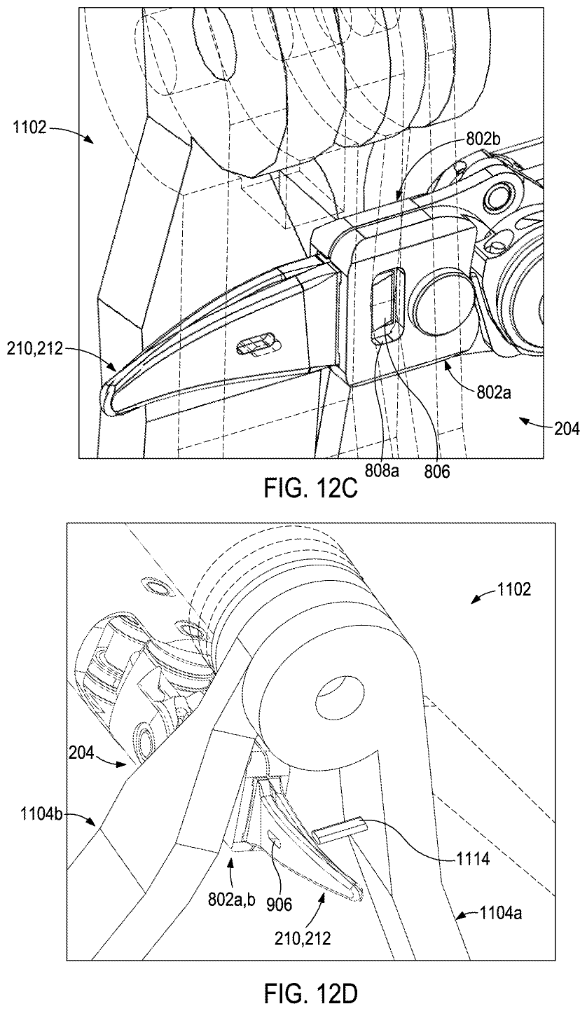

FIGS. 12A-12D depict progressive steps in installing the blades 210, 212 using the tool 1102 of FIGS. 11A-11B, according to one or more embodiments of the present disclosure. Referring first to FIG. 12A, the tool 1102 is depicted with the securing handles 1104a,b in a closed position to grasp onto and otherwise secure the blades 210, 212 therebetween. In FIG. 12A, the first securing handle 1104a is shown in phantom (dashed lines) to enable viewing of the blades 210, 212 secured between the securing handles 1104a,b.

In some embodiments, the tool 1102 may be provided to the user with the blades 210, 212 pre-positioned and otherwise secured between the securing handles 1104a,b, as shown in FIG. 12A. In such embodiments, the user (e.g., surgeon, nurse, bedside assist, etc.) may remove the tool 1102 from its packaging with the blades 210, 212 already properly positioned and ready for attachment to the end effector 204 (FIG. 12B). As illustrated, for example, the securing handles 1104a,b may be rotated to the closed position where the fixture projection 1114 extends through and is otherwise received within aligned (e.g., coaxially) fixture apertures 906 defined in each blade 210, 212.

In FIG. 12B, the tool 1102 is brought into proximity of the end effector 204. The first securing handle 1104a and the first release handle 1106a are shown in FIG. 12B in phantom (dashed lines) to enable viewing of the blades 210, 212. The user may grasp onto the tool 1102 and bring the blades 210, 212 into proximity of the first and second blade holders 802a,b and guide the distal end 904b of the blades 210, 212 into the interior 804 of the blade holders 802a,b.

In FIG. 12C, the user may advance the tool 1102 toward the end effector 204 and simultaneously advance the blades 210, 212 into the interior 804 of the blade holders 802a,b. As the blades 210, 212 are received within the interior 804, the retention mechanisms 806 (only one visible) provided on each blade 210, 212 will engage the opening to the interior 804 on opposite sides, which will cause the retention mechanisms 806 to flex laterally inward to allow the blades 210, 212 to enter the interior 804. The blades 210, 212 are then further advanced proximally B until the retention mechanisms 806 locate the corresponding retention apertures 808a,b (only the first retention aperture 808a is visible) at which point the retention mechanisms 806 are able to spring back to their natural state and be received within the retention aperture 808a,b. Once the retention mechanisms 806 are received within the corresponding retention aperture 808a,b, the blades 210, 212 will be effectively secured within the blade holders 802a,b during operation.

In FIG. 12D, once the blades 210, 212 are properly received within the blade holders 802a,b, as described above, the tool 1102 may then be removed (separated) from the blades 210, 212. More specifically, the user may manually rotate (pivot) the first and second securing handles 1104a,b away from each other and toward an open position. As the securing handles 1104a,b pivot to the open position, the fixture projection 1114 is also removed from the fixture apertures 906 defined in each blade 210, 212. Once the securing handles 1104a,b are sufficiently moved the open position, the tool 1102 may be removed away from the end effector 204 and the surgical tool 200 may then be placed in service.

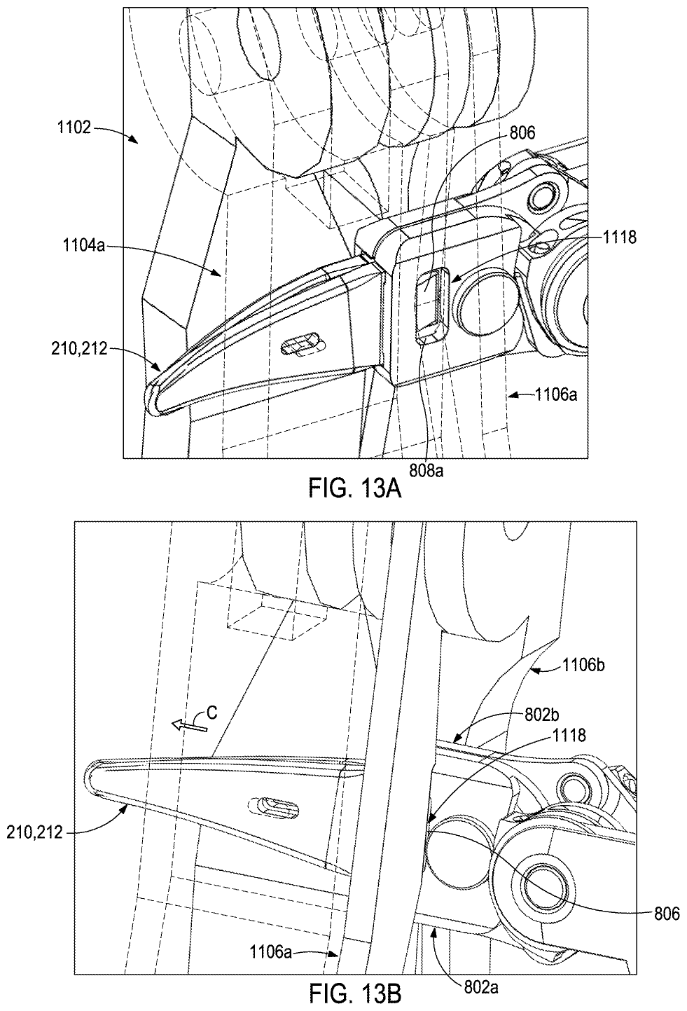

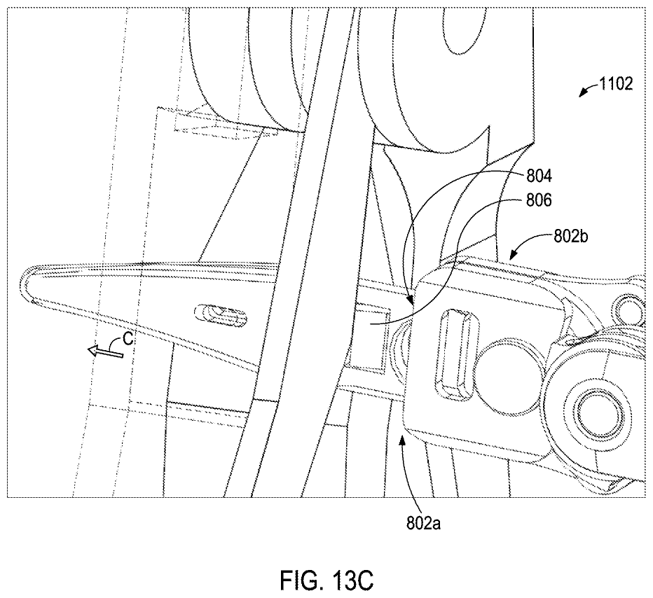

FIGS. 13A-13C depict progressive steps in removing the blades 210, 212 using the tool 1102 of FIGS. 11A-11B, according to one or more embodiments of the present disclosure. Referring first to FIG. 13A, the first securing handle 1104a and the first release handle 1106a are each shown in phantom (dashed lines) to enable viewing of the blades 210, 212. The user may first grasp onto the tool 1102 and bring the tool 1102 into proximity of the end effector 204. The user may then align the securing handles 1104a,b with the blades 210, 212 and, more particularly, align the fixture projection 1114 provided on one or both of the securing handles 1104a,b with the fixture apertures 906 defined in each blade 210, 212. The securing handles 1104a,b may then be moved toward the closed position to receive the fixture projection 1114 within the fixture apertures 906, thereby axially securing the fixture tool 1102 to the blades 210, 212.

The user may then align the first and second release handles 1106a,b (only the first release handle 1106a is visible) with the retention apertures 808a,b (only the first retention aperture 808a is visible). More specifically, the user may align the release projections 1118 of each release handle 1106a,b with the retention apertures 808a,b and manually move (squeeze) the release handles 1106a,b inward to the closed position to engage the release projections 1118 against the retention mechanisms 806. In at least one embodiment, closing the securing handles 1104a,b, blades 210, 212 such that the fixture projection 1114 is received within the fixture apertures 906 will properly align the release handles 1106a,b with the retention apertures 808a,b. In such embodiments, the user need only move the release handles 1106a,b to the closed position to properly engage the release projections 1118 against the retention mechanisms 806.

In FIG. 13B, the release handles 1106a,b are moved to the closed position and the release projections 1118 are engaged against the retention mechanisms 806 on opposing sides of the blades 210, 212 and opposing sides of the blade holders 802a,b. The user may then apply additional pressure on the release handles 1106a,b to force the release projections 1118 against the retention mechanisms 806 and thereby flex retention mechanisms 806 inward and out of engagement with the retention apertures 808a,b (only the first retention aperture 808a is visible). Once the retention mechanisms 806 are disengaged from the corresponding retention apertures 808a,b, the user may then move the blades 210, 212 distally C to remove the blades 210, 212 from the blade holders 802a,b.

In FIG. 13C, tool 1102 is moved distally C by the user. As the blades 210, 212 move distally C, the retention mechanisms 806 remain flexed inward until exiting the interior 804 of the blade holders 802a,b. The blades 210, 212 are then at this point separated from the blade holders 802a,b, and the user may then remove the tool 1102 from the blades 210, 212 and subsequently discard, refurbish, or discard the blades 210, 212, as desired. At this point, if desired, the tool 1102 itself may also be discarded as a one-time use device.

Embodiments disclosed herein include:

A. A surgical tool includes a drive housing, an elongate shaft extending distally from the drive housing, a wrist arranged at a distal end of the shaft, and an end effector operatively coupled to the wrist and including first and second blade pulleys rotatably mounted to the wrist, first and second blade holders mounted to the first and second blade pulleys, respectively, and cooperatively defining an interior, first and second blades mountable to the first and second blade holders, respectively, such that rotational movement of the first and second blade pulleys correspondingly moves the first and second blades between closed and open positions, and a blade retention system that releasably secures the first and second blades to the first and second blade holders, respectively, and allows a user to manually remove and replace the first and second blades.

B. A method of installing blades of an end effector of a surgical tool includes the steps of arranging first and second blades of the end effector adjacent to first and second blade holders of the end effector, the first and second blade holders cooperatively defining an interior, advancing the first and second blades into the interior and thereby causing one or more retention mechanisms forming part of a blade retention system to flex and allow the first and second blades to advance proximally into the interior, and advancing the first and second blades further into the interior until the one or more retention mechanisms to spring back to a natural state and thereby releasably securing the first and second blades to the first and second blade holders, respectively.

Each of embodiments A and B may have one or more of the following additional elements in any combination: Element 1: wherein the blade retention system includes a boss extending laterally outward from a side surface of each blade, and upper and lower retention mechanisms provided on each blade holder and engageable with the boss of a corresponding one of the first and second blades to removably secure the corresponding one of the first and second blades within the interior. Element 2: wherein a longitudinal channel is defined by the first and second blade holders within the interior and is sized to receive the boss of each blade, and wherein the upper and lower retention mechanisms comprise flexible arms provided on upper and lower walls of the longitudinal channel. Element 3: wherein each flexible arm defines an inner profile extending into the longitudinal channel to receive and engage the boss of each blade when the corresponding one of the first and second blades traverses the longitudinal channel, and wherein each flexible arm flexes outward upon engaging the boss. Element 4: wherein the blade retention system includes a first retention mechanism mounted to the first blade and a second retention mechanism mounted to the second blade, each retention mechanism including a flexible leg terminating in a latch feature, a retention aperture defined in each blade holder to receive the latch feature, and a longitudinal recess defined in each blade and sized to accommodate the flexible leg such that a gap is defined between the recess and the flexible leg, wherein the flexible leg flexes into the gap when the blades are advanced within the interior and until the latch feature is received within the retention aperture or exits the interior. Element 5: wherein the latch feature provides an angled proximal end engageable with an inner wall of the interior to flex the flexible leg into the gap. Element 6: wherein the blade retention system includes a first retention mechanism mounted to the first blade holder and a second retention mechanism mounted to the second blade holder, each retention mechanism including a flexible leg that terminates in a latch feature, a retention receptacle defined in each blade and sized to receive the latch feature, and a longitudinal recess defined in each blade and sized to accommodate the flexible leg, wherein the flexible leg is flexed away from the longitudinal recess until the latch feature is received within the retention receptacle or exits the interior. Element 7: wherein the latch feature provides an angled proximal end engageable with the longitudinal recess to flex the flexible leg away from the longitudinal recess. Element 8: wherein each retention mechanism includes a U-shaped body including the flexible leg and an upper leg, and wherein a gap is defined between the flexible and upper legs and the flexible leg is flexed into the gap until the latch feature is received within the retention receptacle or exits the interior. Element 9: wherein each retention mechanism includes a mounting feature received within a mounting receptacle defined in a body of the first or second blade, the flexible leg extending from the mounting feature, and wherein a gap is defined between the flexible leg and an inner wall of a corresponding one of the first and second blade holders to receive the flexible leg when it is flexed away from the longitudinal recess. Element 10: wherein the blade retention system includes a first retention mechanism provided on a shank of the first blade and a second retention mechanism provided on a shank of the second blade, and first and second retention apertures defined by the first and second blade holders, respectively, and sized to receive a corresponding one of the first and second retention mechanisms as the first and second blades are advanced into the interior. Element 11: wherein the first and second retention mechanisms comprise leaf springs. Element 12: further comprising a blade installation and removal tool operable to install and uninstall the first and second blades from the first and second blade holders, the blade installation and removal tool including opposing first and second securing handles mounted to an axle and rotatable about a pivot axis extending through the axle, a fixture projection extending from an inner surface of one or both of the first and second securing handles and configured to be received within aligned fixture apertures defined in each blade, opposing first and second release handles mounted to the axle and rotatable about the pivot axis, and opposing release projections defined on each release handle and configured to align with and extend into the retention apertures to engage and release the first and second retention mechanisms from the first and second retention apertures.

Element 13: wherein a boss extends laterally outward from a side surface of each blade and the one or more retention mechanisms comprise upper and lower flexible arms provided on each blade holder and engageable with the boss of a corresponding one of the first and second blades, and wherein advancing the first and second blades into the interior comprises engaging the boss of the corresponding one of the first and second blades against the upper and lower flexible arms, flexing the upper and lower flexible arms outward to receive the boss of the corresponding one of the first and second blades, bypassing the upper and lower flexible arms with the boss of the corresponding one of the first and second blades, and allowing the upper and lower flexible arms to spring back to the natural state to secure the first and second blades within the interior. Element 14: wherein the one or more retention mechanisms include a first retention mechanism mounted to the first blade and a second retention mechanism mounted to the second blade, each retention mechanism including a flexible leg terminating in a latch feature, and wherein advancing the first and second blades into the interior comprises engaging an angled proximal end of the latch feature against an inner wall of the interior and thereby flexing the flexible leg into a gap defined between the flexible leg and a recess and longitudinal recess defined in each blade, and receiving the latch feature in a retention aperture defined in each blade holder and thereby allowing the flexible leg to spring back to the natural state to secure the first and second blades within the interior. Element 15: wherein the one or more retention mechanisms include a first retention mechanism mounted to the first blade holder and a second retention mechanism mounted to the second blade holder, each retention mechanism including a flexible leg that terminates in a latch feature, and wherein advancing the first and second blades into the interior comprises engaging an angled proximal end of the latch feature against a longitudinal recess defined in each blade and thereby flexing the flexible leg away from the longitudinal recess, and receiving the latch feature in a retention receptacle defined in each blade and thereby allowing the flexible leg to spring back to the natural state to secure the first and second blades within the interior. Element 16: wherein the one or more retention mechanisms include a first retention mechanism provided on a shank of the first blade and a second retention mechanism provided on a shank of the second blade, and wherein advancing the first and second blades into the interior comprises engaging the first and second retention mechanisms against an inner wall of the first and second blade holders, respectively, and thereby flexing the first and second retention mechanisms inward, and locating and receiving the first and second retention mechanisms into first and second retention apertures defined by the first and second blade holders, respectively, and thereby allowing the first and second retention mechanisms to spring back to the natural state to secure the first and second blades within the interior. Element 17:

wherein arranging the first and second blades of the end effector adjacent to the first and second blade holders comprises manually securing the first and second blades between opposing first and second securing handles of a blade installation and removal tool, wherein a fixture projection extends from an inner surface of one or both of the first and second securing handles and is received within aligned fixture apertures defined in each blade, manually moving the first and second blades toward the first and second blade holders while secured between the opposing first and second securing handles, and once the first and second blades are secured within the interior, rotating the opposing first and second securing handles away from each other and thereby removing the fixture projection out of the aligned fixture apertures. Element 18: further comprising manually rotating opposing first and second release handles of the blade installation and removal tool toward each other, wherein opposing release projections are defined on each release handle, aligning the opposing release projections with the first and second retention apertures, engaging the first and second retention mechanisms with the opposing release projections, and releasing the first and second retention mechanisms from the first and second retention apertures by manually squeezing the first and second release handles.

By way of non-limiting example, exemplary combinations applicable to A and B include: Element 1 with Element 2; Element 2 with Element 3; Element 4 with Element 5; Element 6 with Element 7; Element 6 with Element 8; Element 6 with Element 9; Element 10 with Element 11; Element 10 with Element 12; and Element 17 with Element 18.

Therefore, the disclosed systems and methods are well adapted to attain the ends and advantages mentioned as well as those that are inherent therein. The particular embodiments disclosed above are illustrative only, as the teachings of the present disclosure may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope of the present disclosure. The systems and methods illustratively disclosed herein may suitably be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the elements that it introduces. If there is any conflict in the usages of a word or term in this specification and one or more patent or other documents that may be incorporated herein by reference, the definitions that are consistent with this specification should be adopted.

As used herein, the phrase “at least one of” preceding a series of items, with the terms “and” or “or” to separate any of the items, modifies the list as a whole, rather than each member of the list (i.e., each item). The phrase “at least one of” allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

What is claimed is:

1. A surgical tool, comprising:
a drive housing;
an elongate shaft extending distally from the drive housing;
a wrist arranged at a distal end of the shaft; and
an end effector operatively coupled to the wrist and including:
first and second blade pulleys rotatably mounted to the wrist;
first and second blade holders mounted to the first and second blade pulleys, respectively, and cooperatively defining an interior;
first and second blades mountable to the first and second blade holders, respectively, such that rotational movement of the first and second blade pulleys correspondingly moves the first and second blades between closed and open positions; and
a blade retention system that releasably secures the first and second blades to the first and second blade holders, respectively, and allows a user to manually remove and replace the first and second blades.

2. The surgical tool of claim 1, wherein the blade retention system includes:
a boss extending laterally outward from a side surface of each blade; and
upper and lower retention mechanisms provided on each blade holder and engageable with the boss of a corresponding one of the first and second blades to removably secure the corresponding one of the first and second blades within the interior.

3. The surgical tool of claim 2, wherein a longitudinal channel is defined by the first and second blade holders within the interior and is sized to receive the boss of each blade, and wherein the upper and lower retention mechanisms comprise flexible arms provided on upper and lower walls of the longitudinal channel.

4. The surgical tool of claim 3, wherein each flexible arm defines an inner profile extending into the longitudinal channel to receive and engage the boss of each blade when the corresponding one of the first and second blades traverses the longitudinal channel, and
wherein each flexible arm flexes outward upon engaging the boss.

5. The surgical tool of claim 1, wherein the blade retention system includes:
a first retention mechanism mounted to the first blade and a second retention mechanism mounted to the second blade, each retention mechanism including a flexible leg terminating in a latch feature;
a retention aperture defined in each blade holder to receive the latch feature; and
a longitudinal recess defined in each blade and sized to accommodate the flexible leg such that a gap is defined between the recess and the flexible leg,
wherein the flexible leg flexes into the gap when the blades are advanced within the interior and until the latch feature is received within the retention aperture or exits the interior.

6. The surgical tool of claim 5, wherein the latch feature provides an angled proximal end engageable with an inner wall of the interior to flex the flexible leg into the gap.

7. The surgical tool of claim 1, wherein the blade retention system includes:
a first retention mechanism mounted to the first blade holder and a second retention mechanism mounted to the second blade holder, each retention mechanism including a flexible leg that terminates in a latch feature;
a retention receptacle defined in each blade and sized to receive the latch feature; and
a longitudinal recess defined in each blade and sized to accommodate the flexible leg, wherein the flexible leg is flexed away from the longitudinal recess until the latch feature is received within the retention receptacle or exits the interior.

8. The surgical tool of claim 7, wherein the latch feature provides an angled proximal end engageable with the longitudinal recess to flex the flexible leg away from the longitudinal recess.

9. The surgical tool of claim 7, wherein each retention mechanism includes a U-shaped body including the flexible leg and an upper leg, and
wherein a gap is defined between the flexible and upper legs and the flexible leg is flexed into the gap until the latch feature is received within the retention receptacle or exits the interior.

10. The surgical tool of claim 7, wherein each retention mechanism includes a mounting feature received within a mounting receptacle defined in a body of the first or second blade, the flexible leg extending from the mounting feature, and
wherein a gap is defined between the flexible leg and an inner wall of a corresponding one of the first and second blade holders to receive the flexible leg when it is flexed away from the longitudinal recess.

11. The surgical tool of claim 1, wherein the blade retention system includes:
a first retention mechanism provided on a shank of the first blade and a second retention mechanism provided on a shank of the second blade; and
first and second retention apertures defined by the first and second blade holders, respectively, and sized to receive a corresponding one of the first and second retention mechanisms as the first and second blades are advanced into the interior.

12. The surgical tool of claim 11, wherein the first and second retention mechanisms comprise leaf springs.

13. The surgical tool of claim 11, further comprising a blade installation and removal tool operable to install and uninstall the first and second blades from the first and second blade holders, the blade installation and removal tool including:
opposing first and second securing handles mounted to an axle and rotatable about a pivot axis extending through the axle;
a fixture projection extending from an inner surface of one or both of the first and second securing handles and configured to be received within aligned fixture apertures defined in each blade;
opposing first and second release handles mounted to the axle and rotatable about the pivot axis; and
opposing release projections defined on each release handle and configured to align with and extend into the retention apertures to engage and release the first and second retention mechanisms from the first and second retention apertures.

14. A method of installing blades of an end effector of a surgical tool, comprising:
arranging first and second blades of the end effector adjacent to first and second blade holders of the end effector, the first and second blade holders cooperatively defining an interior;
advancing the first and second blades into the interior and thereby causing one or more retention mechanisms forming part of a blade retention system to flex and allow the first and second blades to advance proximally into the interior; and
advancing the first and second blades further into the interior until the one or more retention mechanisms to spring back to a natural state and thereby releasably securing the first and second blades to the first and second blade holders, respectively.

15. The method of claim 14, wherein a boss extends laterally outward from a side surface of each blade and the one or more retention mechanisms comprise upper and lower flexible arms provided on each blade holder and engageable with the boss of a corresponding one of the first and second blades, and wherein advancing the first and second blades into the interior comprises:
engaging the boss of the corresponding one of the first and second blades against the upper and lower flexible arms;
flexing the upper and lower flexible arms outward to receive the boss of the corresponding one of the first and second blades;
bypassing the upper and lower flexible arms with the boss of the corresponding one of the first and second blades; and
allowing the upper and lower flexible arms to spring back to the natural state to secure the first and second blades within the interior.

16. The method of claim 14, wherein the one or more retention mechanisms include a first retention mechanism mounted to the first blade and a second retention mechanism mounted to the second blade, each retention mechanism including a flexible leg terminating in a latch feature, and wherein advancing the first and second blades into the interior comprises:
engaging an angled proximal end of the latch feature against an inner wall of the interior and thereby flexing the flexible leg into a gap defined between the flexible leg and a recess and longitudinal recess defined in each blade; and
receiving the latch feature in a retention aperture defined in each blade holder and thereby allowing the flexible leg to spring back to the natural state to secure the first and second blades within the interior.

17. The method tool of claim 14, wherein the one or more retention mechanisms include a first retention mechanism mounted to the first blade holder and a second retention mechanism mounted to the second blade holder, each retention mechanism including a flexible leg that terminates in a latch feature, and wherein advancing the first and second blades into the interior comprises:
engaging an angled proximal end of the latch feature against a longitudinal recess defined in each blade and thereby flexing the flexible leg away from the longitudinal recess; and
receiving the latch feature in a retention receptacle defined in each blade and thereby allowing the flexible leg to spring back to the natural state to secure the first and second blades within the interior.

18. The method of claim 14, wherein the one or more retention mechanisms include a first retention mechanism provided on a shank of the first blade and a second retention mechanism provided on a shank of the second blade, and wherein advancing the first and second blades into the interior comprises:
engaging the first and second retention mechanisms against an inner wall of the first and second blade holders, respectively, and thereby flexing the first and second retention mechanisms inward; and
locating and receiving the first and second retention mechanisms into first and second retention apertures defined by the first and second blade holders, respectively, and thereby allowing the first and second retention mechanisms to spring back to the natural state to secure the first and second blades within the interior.

19. The method of claim 14, wherein arranging the first and second blades of the end effector adjacent to the first and second blade holders comprises:
manually securing the first and second blades between opposing first and second securing handles of a blade installation and removal tool, wherein a fixture projection extends from an inner surface of one or both of the first and second securing handles and is received within aligned fixture apertures defined in each blade;
manually moving the first and second blades toward the first and second blade holders while secured between the opposing first and second securing handles; and
once the first and second blades are secured within the interior, rotating the opposing first and second securing handles away from each other and thereby removing the fixture projection out of the aligned fixture apertures.

20. The method of claim 19, further comprising:
manually rotating opposing first and second release handles of the blade installation and removal tool toward each other, wherein opposing release projections are defined on each release handle;
aligning the opposing release projections with the first and second retention apertures;
engaging the first and second retention mechanisms with the opposing release projections; and
releasing the first and second retention mechanisms from the first and second retention apertures by manually squeezing the first and second release handles.
